(19) United States
(12) Patent Application Publication
(10) Pub. No.: US 2010/0113379 A1
(43) Pub. Date: May 6, 2010

(54) cAMP DEPENDENT INDUCTION OF AUTOPHAGY

(76) Inventors: David Rubinsztein, Cambridge (GB); Sovan Sarkar, Cambridge (GB); Andrea Williams, Cambridge (GB)

Correspondence Address: KLARQUIST SPARKMAN, LLP, 121 SW SALMON STREET, SUITE 1600, PORTLAND, OR, 97204, US

(21) Appl. No.: 12/527,404

(22) PCT Filed: Feb. 14, 2008
(86) PCT No.: PCT/GB08/00501

§ 371 Date: Aug. 14, 2009

(30) Foreign Application Priority Data
Feb. 15, 2007 (GB) 0702972.1

(51) Int. Cl. A61K 31/7076; A61K 31/4168; A61K 31/506; A61K 31/135; A61K 31/436; C12Q 1/68; A61P 31/04; A61P 31/12; A61P 25/16; A61P 25/28

(52) U.S. Cl. 514/46; 514/401; 514/275; 514/654; 514/291; 435/6

(57) ABSTRACT

The present invention relates to the induction of autophagy via mTOR independent pathways which modulated intracytosolic cAMP levels. In some embodiments, autophagy may be induced in a cell by reducing intracytosolic cAMP levels, for example using a cAMP antagonist, such as clonidine. This may be useful, for example in the treatment of neurodegenerative disorders or pathogen infections.

This invention relates to the induction of autophagy in cells. This is useful in clearing intracellular protein aggregates, for example in the treatment of a neurodegenerative disorder or pathogen infection.

The autophagy-lysosomal and ubiquitin-proteasome pathways are major routes for protein and organelle clearance in eukaryotic cells. The narrow pore of the proteasome barrel precludes clearance of large membrane proteins and protein complexes (including oligomers and aggregates), but mammalian lysosomes can degrade protein complexes and organelles by macroautophagy, which is generally referred to as autophagy. It involves the formation of double membrane structures called autophagosomes around a portion of cytosol. These fuse with lysosomes where their contents are degraded. Autophagy can be induced by several conditions, including starvation, and is regulated by a number of protein kinases, the best characterised being the mammalian target of rapamycin (mTOR).

Autophagy induction may represent a tractable therapeutic strategy for neurodegenerative disorders caused by aggregate-prone intracytosolic proteins, including Huntington's disease (HD), an autosomal-dominant neurodegenerative disorder caused by a CAG trinucleotide repeat expansion (>35 repeats), which encodes an abnormally long polyglutamine (polyQ) tract in the N-terminus of the huntingtin protein (1). Mutant huntingtin toxicity is thought to be exposed after it is cleaved to form N-terminal fragments comprising the first 100-150 residues with the expanded polyQ tract, which are also the toxic species found in aggregates/inclusions. Thus, HD pathogenesis is frequently modelled with exon 1 fragments containing expanded polyQ repeats which cause aggregate formation and toxicity in cell models and in vivo. The polyQ mutation in HD raises intracellular calcium (Ca²⁺) levels resulting in enhanced calpain activity, and this has been proposed as an important disease mechanism (2). One possibility is that calpains enhance mutant huntingtin cleavage/processing.

In addition to mutant huntingtin, autophagy also regulates the clearance of other aggregate-prone disease-causing proteins, like those causing spinocerebellar ataxias types 1 and 3, forms of tau (causing fronto-temporal dementias) and the A53T and A30P α-synuclein mutants (which cause familial Parkinson's disease (PD)) (3-8). Autophagy induction reduces both soluble mutant huntingtin levels and inclusion frequencies, and attenuates its toxicity in cell, Drosophila and mouse models of HD (3, 9).

Autophagy induction may also be a valuable strategy in the treatment of infectious diseases, including tuberculosis and group A streptococcal infections (10-12) and may protect against cell death in certain contexts (13). Currently, the only suitable pharmacological strategy for up-regulating autophagy in mammalian brains is to use rapamycin, which inhibits mTOR (9). However, rapamycin is an immunosuppressant, precluding its use in diseases like tuberculosis. The mechanism by which mTOR regulates autophagy remains unclear and mTOR is known to control several cellular processes besides autophagy (14), probably contributing to the complications seen with long-term use of rapamycin. Thus, we sought to identify novel pathways and therapeutic agents that enhance autophagy.

The present inventors have discovered that autophagy is regulated by mTOR independent pathways which involve modulation of the level of intracytosolic cAMP. Regulation of these pathways allows for the induction of autophagy, for example in the treatment of a neurodegenerative disorder or pathogen infection.

One aspect of the invention provides a method of inducing or promoting autophagy in a cell comprising:
inhibiting or reducing the activity of the cAMP/EPAC/PLC pathway in said cell.

Other aspects of the invention provide (i) an inhibitor or antagonist of the cAMP/EPAC/PLC pathway in a cell for use in the induction of autophagy in the cell and (ii) the use of an inhibitor or antagonist of the cAMP/EPAC/PLC pathway in a cell in the manufacture of a medicament for use in the induction of autophagy in the cell.

The cAMP/EPAC/PLC pathway modulates the release of Ca²⁺ from the ER into the cytosol, thereby increasing the level of cytosolic Ca²⁺, in response to the level of 3′,5′-cyclic adenosine monophosphate (cAMP) in the cell.

The cAMP/EPAC/PLC pathway comprises the components Epac1, RapB and PLC-ε.

EPAC1 (also known as RAPGEF3 or Rap guanine nucleotide exchange factor (GEF) 3) is encoded by the nucleic acid sequence of database entry NM_{—}006105.3 GI: 45269150 or an allelic variant thereof and may have the amino acid sequence of database entry NP_{—}006096.2 GI: 20070215.

Rap2B is encoded by the nucleic acid sequence of database entry NM_{—}002886.2 GI: 38201689 or an allelic variant thereof and may have the amino acid sequence of database entry NP_{—}002877.2 GI: 38201690.

PLC-ε (also known as PLCE1 or phospholipase C, epsilon 1) is encoded by the nucleic acid sequence of database entry NM_{—}016341.3 GI: 117168249 or an allelic variant thereof and may have the amino acid sequence of database entry NP_{—}057425.3 GI: 117168250.

Inhibition or reduction in the activity of the cAMP/EPAC/PLC pathway in a cell reduces the level of Ca²⁺ in the cytosol and thereby induces autophagy in the cell.

In some embodiments, the activity of the cAMP/EPAC/PLC pathway in a cell is modulated, e.g. reduced or inhibited, by altering, e.g. reducing, the amount of cytosolic 3′, 5′ cyclic adenosine monophosphate (cAMP) in the cell.

The level or amount of cytosolic cAMP may be reduced by contacting the cell with a cAMP antagonist. A cAMP antagonist reduces cytosolic cAMP levels, for example, by increasing the depletion of cAMP, inhibiting the synthesis of cAMP, promoting the transport of cAMP out of the cytosol, activating or enhancing Gi/cAMP pathways and/or inhibiting Gs/cAMP pathways.

A cAMP antagonist may for example interact with cAMP-coupled receptors on the cell surface to modulate cAMP levels in the cytosol of the cell.

In some embodiments, the level or amount of cAMP in the cell may be reduced by activating one or more cellular factors, for example one or more components of the G₁ signalling pathway, which inhibits or reduces the activity of adenylate cyclase.

Suitable cAMP antagonists include clonidine, rilmendine, tyramine morphine, baclofen, G protein receptor-derived peptides (Taylor et al Cell Signal 1994 6 841-849), mastoparan (Higashijima et al J. Biol Chem 1988 263 6491-6494; Higashijima et al J. Biol Chem 1990 265 14176-14186), propranolol, bupivacain (Hagelu″ ken, A et al Biochem. Pharmacol. 1994, 47, 1789-1795), quinine, aspartame (Naim, M. Et al Biochem. J. 1994, 297, 451-454), N-dodecyl lysinamide and FUB 86 (Leschke, C. et al J. Med. Chem. 1997, 40, 3130-3139; Breitweg-Lehmann, E. Mol. Pharmacol. 2002, 61, 628-636; Mousli, M. et al Trends Pharmacol. Sci. 1990, 11, 358-362).

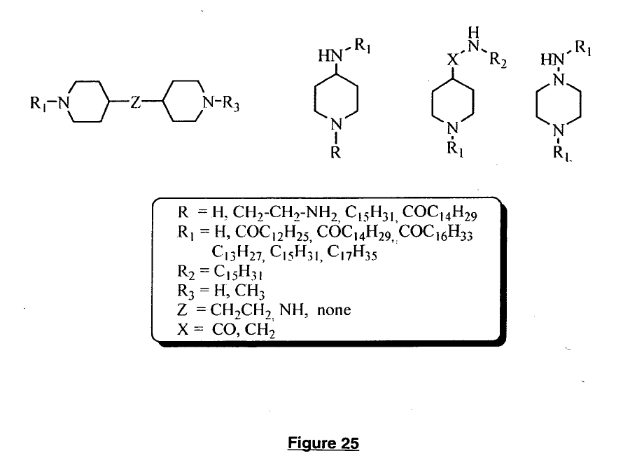

Other cAMP antagonists suitable for use as described herein are shown in FIG. 24 (Melchiorre, C et al. J. Med. Chem. 2001, 44, 4035-4038) and FIG. 25 (Manetti et al et al J. Med. Chem. (2005) 48 6491-6503). Clonidine is an adrenergic agonist which has the IUPAC name N-(2,6-dichlorophenyl)-4,5-dihydro-1H-imidazol-2-amine (CAS 4205-90-7). Clonidine is commercially available (e.g. Catapres®, Dixarit®, Boehringer Ingelheim) as an anti-hypertensive agent and also used in the treatment of pain and opioid detoxification. (Khan, Z. P., et al. Anaesthesia 54, 146, (1999), Head, G. A., et al. J. Auton. Derv. Syst. 72, 163 (1998)).

Rilmenidine has the IUPAC name 2-[N-(Dicyclopropylmethyl)amino]oxazoline hemifuramate (CAS 54187-04-1) (Chan, C. K. et al. J. Pharmacol. Exp. Ther. 276, 411, (1998); Takada, K. et al. Br. J. Pharmacol. 120, 1575, (1997); Ozog, M., et al., J. Neurochem. 71, 1429-1435, (1998) and is commercially available as an anti-hypertensive agent (HYPERIUM®, Servier).

Tyramine has the IUPAC name 4-hydroxy-phenethylamine (CAS 51-67-2). (Bunzow, J. R., et al. Mol. Pharmacol. 60, 1181, (2001); K. Matsuoka et al. Chem. Pharm. Bull. 27, 2345, (1979)) and is available from commercial sources (e.g. Sigma-Aldrich).

Morphine has the IUPAC name 7,8-didehydro-4,5-epoxy-17-methylmorphinan-3,6-diol (CAS 57-27-2) and is available from commercial sources (e.g. Sigma-Aldrich).

Baclofen has the IUPAC name 4-amino-3-(4-chlorophenyl)-butanoic acid (CAS 1134-47-0) and is available from commercial sources (e.g. Sigma-Aldrich).

Other suitable cAMP antagonists may be isomers, salts, solvates, chemically protected forms, and prodrugs of any of the above compounds.

In some embodiments, the level or amount of cAMP in the cell may be reduced by inhibiting the activity of a cellular factor, for example a member of a Gₛ signalling pathway, which stimulates, activates or increases the activity of adenylate cyclase. Examples of such cellular factors include Gₛα and its ligand, pituitary adenylate cyclase-activating polypeptide (PACAP) or other members of the PACAP/Glucagon superfamily.

Other members of the PACAP/Glucagon superfamily include secretin, peptide histidine methionine (PHM), vasoactive intestinal peptide (VIP), glucagon, glucagon like peptide-1 (GLP-1), GLP-2, glucose dependent insulinotropic polypeptide (GIP), GH releasing factor (GRF) and PACAP related peptide (PRP) (Sherwood et al. Endocrine reviews 2000 December; 21(6):619-70).

Gₛα is the alpha subunit of heterotrimeric G-proteins. The amino acid sequence of human Gₛα (also known as adenylate cyclase-stimulating G alpha protein or GNAS; NCBI GeneID: 2778) is available to the public under GenBank entry NP_{—}000507.1 GI: 4504047 and the encoding nucleic acid under reference NM_{—}000516.4 GI: 117938757.

The natural ligand of Gₛα is pituitary adenylate cyclase-activating polypeptide (PACAP), which interacts with Gₛα to increase the activity of adenylate cyclase in target cells, thereby increasing the level of cAMP. The amino acid sequence of PACAP (also known as ADCYAP1 or adenylate cyclase activating polypeptide 1 NCBI GeneID 116) is available under GenBank entry NP_{—}001108.1 GI: 4501921 and the encoding nucleic acid under entry NM_{—}001117.2 GI: 10947062.

Methods of measuring the activity of Gₛα and/or PACAP are well-known in the art, and include for example, monitoring levels of cAMP in a cell. cAMP levels may be assayed by a range of techniques as reviewed in Post et al Methods Mol Biol 2000; 126:363-74.

The activity of Gₛα and/or PACAP may be inhibited by contacting the cell with an antagonist of Gₛα or PACAP. Examples of Gₛα/PACAP antagonists include suramin and suramin analogues, such as NF449 and NF503.

Suramin has the IUPAC name 8-[[4-methyl-3-[[3-[[3-[[2-methyl-5-[(4,6,8-trisulfonaphthalen-1-yl)carbamoyl]phenyl]carbamoyl]phenyl]carbamoylamino]benzoyl]amino]benzoyl]amino]naphthalene-1,3,5-trisulfonic acid (CAS 129-46-4) and is commercially available (e.g. Sigma-Aldrich).

NF449 is a suramin analogue which has the IUPAC name 4,4′,4″, 4′″-(carbonylbis(imino-5,1,3-benzenetriylbis(carbonylimino)))tetrakis-benzene-1,3-disulfonic acid (see for example Hohenegger, M., Proc. Natl. Acad. Sci. USA 95, 346, (1998), Rettinger, J., et al Neuropharmacology 48, 461-468, (2005)) and is commercially available (e.g. Sigma-Aldrich).

NF503 is a suramin analogue which has the IUPAC name 4,4′-[carbonylbis [imino-3,1-phenylene-(2,5 benzimidazolylene) carbonylimino]]bis-benzenesulfonate (Hohenegger, M., Proc. Natl. Acad. Sci. USA 95, 346, (1998)).

Other suitable agents may be isomers, salts, solvates, chemically protected forms, and prodrugs of Suramin, NF449 and NF503.

In some embodiments, the amount of cAMP in the cytosol of the cell may be reduced by directly inhibiting the activity of adenylate cyclase, for example by contacting the cell with an adenylate cyclase antagonist.

Adenylate cyclase (EC 4.6.1.1) catalyzes the conversion of ATP to 3′,5′-cyclic AMP (cAMP) and pyrophosphate. Human adenylate cyclases include ADCY1 to ADCY9. The amino acid and encoding nucleic acid sequences of these adenylate cyclases are well known in the art. For example, ADCY1 (adenylate cyclase 1 GeneID: 10) is encoded by the nucleic acid sequence of database entry NM_{—}021116.1 GI: 31083192 and may have the amino acid sequence of database entry NP_{—}066939.1 GI: 31083193.

Methods of determining adenylate cyclase activity are well-known in the art (see, for example, Post et al Methods Mol. Biol. 2000 126 363-374).

Examples of adenylate cyclase antagonists include cAMP analogs such as 2′5′ dideoxyadenosine, 2′5′ dideoxyadenosine-3′ monophosphate, 2′5′ dideoxyadenosine-3′ diphosphate and 2′5′ dideoxyadenosine 3′ triphosphate (see, for example, Desaubry et al JBC 271 (1996) 2380-2382), and isomers, salts, solvates, chemically protected forms, and prodrugs of these compounds.

In other embodiments, the activity of the cAMP/EPAC/PLC pathway is inhibited in a cell by directly inhibiting the activity of a component of the pathway, for example by contacting the cell with an antagonist of the component.

Components of the cAMP/EPAC/PLC pathway include Epac1, Rap2B and PLC-ε, which are described in more detail above.

One class of antagonist of a target protein described herein, for example, adenylate cyclase, Gₛα, PACAP or a component of the cAMP/EPAC/PLC pathway such as Epac1, Rap2B and PLC-ε, can be derived from sequence of the target protein. The sequences of all these target proteins are publicly available. Membrane permeable peptide fragments of from 5 to 40 amino acids, for example, from 6 to 10 amino acids may be tested for their ability to modulate activity of the component.

Peptides can also be generated wholly or partly by chemical synthesis according to well-established, standard liquid or, preferably, solid-phase peptide synthesis methods, general descriptions of which are broadly available (see, for example, in J. M. Stewart and J. D. Young, Solid Phase Peptide Synthesis, 2nd edition, Pierce Chemical Company, Rockford, Ill. (1984), in M. Bodanzsky and A. Bodanzsky, The Practice of Peptide Synthesis, Springer Verlag, New York (1984); and Applied Biosystems 430A Users Manual, ABI Inc., Foster City, Calif.). Peptides may be prepared in solution, by the liquid phase method or by any combination of solid-phase, liquid phase and solution chemistry, e.g. by first completing the respective peptide portion and then, if desired and appropriate, after removal of any protecting groups being present, by introduction of the residue X by reaction of the respective carbonic or sulphonic acid or a reactive derivative thereof. The modulatory properties of a peptide may be enhanced by the addition of one of the following groups to the C terminal: chloromethyl ketone, aldehyde and boronic acid. These groups are transition state analogues for serine, cysteine and threonine proteases. The N terminus of a peptide fragment may be blocked with carbobenzyl to inhibit aminopeptidases and improve stability (Proteolytic Enzymes 2nd Ed, Edited by R. Beynon and J. Bond Oxford University Press 2001).

Other candidate modulator compounds may be based on modelling the 3-dimensional structure of a polypeptide or peptide fragment and using rational drug design to provide potential inhibitor compounds with particular molecular shape, size and charge characteristics. Many suitable approaches for the rational design of drugs to a specified protein target are known in the art.

Antibody molecules directed to the target protein may form a further class of putative antagonist. Candidate antibodies may be characterised and their binding regions determined to provide single chain antibodies and frayments thereof which are responsible for blocking or inhibiting activity. Suitable antagonistic antibody molecules may be produced by screening libraries of antibody antigen binding domains displayed on virus particles to identify an antibody antigen binding domain which decreases or inhibits the activity of the target protein.

Another class of antagonist of a target protein described herein may be a polynucleotide which reduces the expression of the component, for example a sense, anti-sense or RNAi polynucleotide, which reduces or abrogates expression of the component. The use of these approaches to down-regulate gene expression is now well-established in the art.

Anti-sense oligonucleotides may be designed to hybridise to the complementary sequence of nucleic acid, pre-mRNA or mature mRNA encoding target protein, thereby interfering with the production of target protein so that its expression is reduced or completely or substantially completely prevented. In addition to targeting coding sequence, anti-sense techniques may be used to target control sequences of a gene, e.g. in the 5′ flanking sequence, whereby the antisense oligonucleotides can interfere with expression control sequences. The construction of antisense sequences and their use is described for example in Peyman and Ulman (1990) Chemical Reviews 90:543-584 and Crooke (1992) Ann. Rev. Pharmacol. Toxicol. 32:329-376. Oligonucleotides may be generated in vitro or ex vivo for administration or anti-sense RNA may be generated in vivo within cells in which down-regulation is desired. Thus, double-stranded DNA may be placed under the control of a promoter in a “reverse orientation” such that transcription of the anti-sense strand of the DNA yields RNA which is complementary to normal mRNA transcribed from the sense strand of the target gene.

The complete sequence corresponding to the coding sequence in reverse orientation need not be used. For example, fragments of sufficient length may be used. It is a routine matter for the person skilled in the art to screen fragments of various sizes and from various parts of the coding or flanking sequences of a gene to optimise the level of anti-sense inhibition. It may be advantageous to include the initiating methionine ATG codon, and perhaps one or more nucleotides upstream of the initiating codon.

Preferably, the anti-sense nucleic acids are chosen among the polynucleotides of 15-200 bp long that are complementary to the 5′ end of a nucleic acid encoding a target protein. A suitable fragment may for example have about 14-23 nucleotides, e.g. about 15, 16 or 17.

Preferred anti-sense nucleic acids are complementary to a sequence of an mRNA encoding a target protein that contains the translational initiation codon ATG. However, the antisense nucleic acid may also be complementary to a sequence in the 3′ or 5′ un-translated regions or to sequences in the splice sites of the pre-mRNA precursor.

Anti-sense oligonucleotides may be deoxyribonucleotides, ribonucleotides or protein nucleic acids and may optionally comprise chemical modifications that prevent degradation by endogenous nucleases such as phosphorothioate oligonucleotides or morpholino oligonucleotides (Heasman et al (2000) Developmental Biology, 222:124-134).

Examples of anti-sense molecules suitable for use as cAMP antagonists are described in Ghelkardini et al N. S. Arch. Pharmacol. 2002, 365, 1-7, Galeotti, N. et al. Neuroscience 2002, 109, 811-818; Sanchez-Blazquez, P. et al Life Sci. 1993, 53, PL381-PL386; Raffa, R. B. et al Eur. J. Pharmacol. 1994, 258, 5-7; Raffa, R. B. et al Life Sci. 1996, 58, 77-80).

An alternative to anti-sense is to use a copy of all or part of the target gene inserted in sense, that is the same, orientation as the target gene, to achieve reduction in expression of the target gene by co-suppression; Angell & Baulcombe (1997) The EMBO Journal 16 12:3675-3684; and Voinnet & Baulcombe (1997) Nature 389 553). Double stranded RNA (dsRNA) has been found to be even more effective in gene silencing than either sense or antisense strands alone (Fire A. et al Nature 391 (1998)). dsRNA mediated silencing is gene specific and is often termed RNA interference (RNAi).

RNA interference is a two-step process. First, dsRNA is cleaved within the cell to yield short interfering RNAs (siRNAs) of about 21-23 nt lengths with 5′ terminal phosphate and 3′ short overhangs (−2 nt). The siRNAs target the corresponding mRNA sequence specifically for destruction (Zamore P. D. Nature Structural Biology, 8, 9, 746-750, (2001)

RNAi may also be efficiently induced using chemically synthesized siRNA duplexes of the same structure with 3′-overhang ends (Zamore P D et al Cell, 101, 25-33, (2000)). Synthetic siRNA duplexes have been shown to specifically suppress expression of endogenous and heterologeous genes in a wide range of mammalian cell lines (Elbashir S M. et al. Nature, 411, 494-498, (2001)).

Another possibility is that nucleic acid is used which on transcription produces a ribozyme, able to cut nucleic acid at a specific site—thus also useful in influencing gene expression. Background references for ribozymes include Kashani-Sabet and Scanlon, 1995, Cancer Gene Therapy, 2(3): 213-223, and Mercola and Cohen, 1995, Cancer Gene Therapy, 2(1), 47-59.

Thus, in some embodiments, a cAMP antagonist may comprise a nucleic acid molecule which consists of all or part of the coding sequence of a target protein and/or the complement thereof. Target proteins and their coding sequences are described above. The type of suppression will also determine whether the molecule is double or single stranded and whether it is RNA or DNA. The skilled person is readily able design suitable nucleic acids for suppression of expression based on the coding sequence of a target protein.

Such a molecule may suppress the expression of a target protein and may comprise a sense or anti-sense target protein coding sequence or may be a target protein specific ribozyme, according to the type of suppression to be employed.

Agonists and antagonists suitable for use as described herein may be specific antagonists of target proteins, or may also act on other related proteins (i.e. non-specific antagonists).

The present inventors have also shown that ATP-sensitive K⁺ channel agonists are useful in reducing the level of cytosolic Ca²⁺ in a cell, thereby inducing autophagy.

Other aspects of the invention provide a method of inducing or promoting autophagy in a cell comprising:
increasing the activity of a ATP-sensitive K⁺ channel in said cell.

The activity of an ATP-sensitive K⁺ channel may be increased by contacting the cell with an ATP-sensitive K⁺ channel agonist.

Other aspects of the invention provide (i) an ATP-sensitive K⁺ channel agonist for use in the induction of autophagy in the cell and (ii) the use of an ATP-sensitive K⁺ channel agonist in the manufacture of a medicament for use in the induction of autophagy in the cell.

ATP-sensitive K⁺ channels mediate the transport of K⁺ across the cell membrane in response to ATP. ATP-sensitive K(+) (K(ATP)) channels comprise the pore-forming subunit (Kir6.1 or Kir6.2) and the regulatory subunit sulfonylurea receptors (SUR1 or SUR2).

Kir6.1 (GeneID: 3764) may have the sequence of GenBank reference Swiss protein accession number NP_{—}004973.1 GI: 4826802 and be encoded by the nucleic acid of reference NM_{—}004982.2 GI: 25121968.

Kir6.2 (GeneID: 3767) may have the sequence of GenBank reference Swiss protein accession number NP_{—}000516.3 GI: 62388888 and be encoded by the nucleic acid of reference NM_{—}000525.3 GI: 62388887.

SUR1 (GeneID: 6833) may have the sequence of GenBank reference Swiss protein accession number NP_{—}000343.2 GI: 118582255 and be encoded by the nucleic acid of reference NM_{—}000352.3 GI: 118582254.

SUR2 (GeneID: 10060) may have the sequence of GenBank reference Swiss protein accession number NP_{—}005682.2 GI: 110832835 and be encoded by the nucleic acid of reference NM_{—}005691.2 GI: 110832834.

An ATP-sensitive K⁺ channel agonist is a compound which activates or increases the activity of ATP-sensitive K⁺ channels. Examples of ATP-sensitive K⁺ channel agonists include minoxidil, pinacidil, cromakalim and isomers, salts, solvates, chemically protected forms, and prodrugs thereof. Other examples of ATP-sensitive K⁺ channel agonists are described in Cecchetti V et al Curr Top Med. Chem. 2006; 6(10):1049-68.

Minoxidil has the IUPAC name 6-(1-Piperidinyl)-2,4-pyrimidinediamine 3-oxide (CAS 38304-91-5) and has previously been used as a vasodilator and hair growth stimulant (van der Velden, J., Cell. Mol. Life. Sci. 55, 788, (1999), Malhi, H., et al., J. Biol. Chem. 275, 26050-26057, (2000), Kourie, J. I., et al. J. Membr. Biol. 164, 47-58, (1998), Kourie, J. I., et al., J. Membr. Biol. 164, 47-58, (1998) Br. J. Pharmacol. 123, 1395-1402, (1998)).

Pinacidil has the IUPAC name (±)-N-Cyano-N′-4-pyridinyl-N″-(1,2,2-trimethylpropyl)guanidine (CAS 85371-64-8) and has previously been used as a hypertensive (Gojkovic-Bukarica, L., et al. Fundam. Clin. Pharmacol. 13, 527-534, (1999)) and is commercially available (e.g. Sigma-Aldrich).

Cromakalim has the IUPAC name (±)-trans-6-Cyano-3,4-dihydro-2,2-dimethyl-4-(2-oxopyrrolidin-1-yl)-2H-1-benzopyran-3-ol (CAS 94470-67-4) (Spinelli, W., et al. Eur. J. Pharmacol. 179, 243, (1990), Lijnen, P., et al. Eur. J. Clin. Pharmacol. 37, 609, (1989)) and is commercially available (e.g. Sigma-Aldrich).

Methods of determining ATP-sensitive K⁺ channel activity are well-known in the art and include electrophysiological techniques such as patch clamping.

The pathway described above is shown herein to be independent of the mTOR dependent pathway described previously (see, for example, WO2004 GB00690). An additive effect may be observed by inducing autophagy through the mTOR dependent pathway in addition to the cAMP/EPAC/PLC mediated pathway described herein.

In some preferred embodiments, in addition to inducing autophagy as described above as described above, the cell may be contacted with an mTOR inhibitor.

The mTOR inhibitor may be contacted with the cell sequentially or simultaneously with the cAMP/EPAC/PLC pathway antagonist.

Suitable mTOR inhibitors include rapamycin macrolides such as rapamycin or derivatives or analogues thereof, including O-alkyl rapamycins, carboxylic acid esters, amide esters, carbamates, fluorinated esters, acetals and silyl ethers, dialdehydes, oximes, hydrazones, hydroxylamines and enols, and CCI-779 and RAD-001.

Rapamycin and its derivatives and analogues are lactam macrolides. A macrolide is a macrocyclic lactone, for example a compound having a 12-membered or larger lactone ring. Lactam macrolides are macrocyclic compounds which have a lactam (amide) bond in the macrocycle in addition to a lactone (ester) bond.

Rapamycin is produced by Streptomyces hygroscopicus, and has the structure shown below.

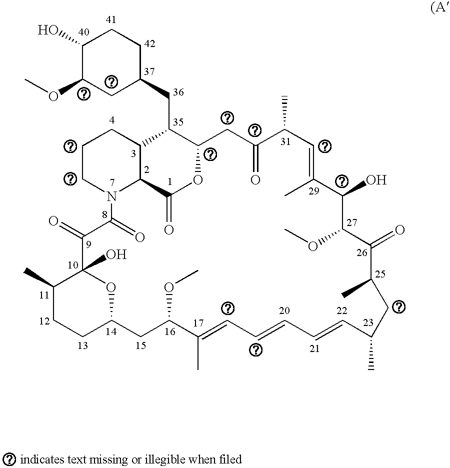

See, e.g., McAlpine J. B. et al. J. Antibiotics (1991) 44: 688; Schreiber, S. L. et al. J. Am. Chem. Soc. (1991) 113:7433; U.S. Pat. No. 3,929,992.

One group of rapamycin analogues are 40-O-substituted derivatives of rapamycin having the structure set out below;

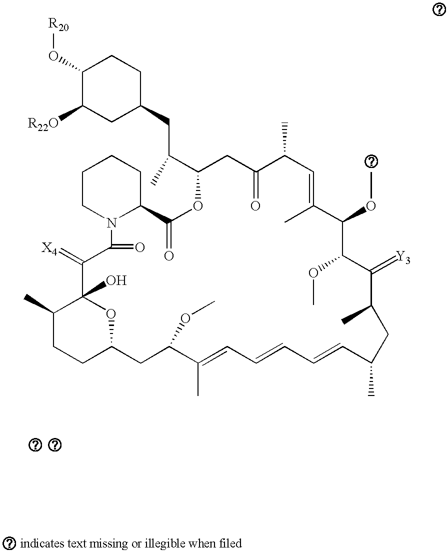

wherein; X₄ is (H,H) or O; Y₃ is (H₂OH) or O; R₂₀ and R₂₁ are independently selected from H, alkyl, arylalkyl, hydroxyalkyl, dihydroxyalkyl, hydroxyalkoxycarbonylalkyl, hydroxyalkylaryalkyl, dihydroxyalkylarylalkyl, acyloxyalkyl, aminoalkyl, alkylaminoalkyl, alkoxycarbonylaminoalkyl, acylaminoalkyl, arylsulfonamidoalkyl, allyl, dihydroxyalkylallyl, dioxolanylallyl, dialkyl-dioxolanylalkyl, di(alkoxycarbonyl)-triazolyl-alkyl and hydroxyalkoxy-alkyl;
wherein “alk-” or “alkyl” refers to C₁₋₆alkyl, branched or linear, preferably C₁₋₃ alkyl; “aryl” is phenyl or tolyl; and acyl is a radical derived from a carboxylic acid; and;
R₂₂ is methyl or R₂₂ and R₂₀ together form C₂₋₆ alkyl; provided that R₂₀ and R₂₁ are not both H; and hydroxyalkoxyalkyl is other than hydroxyalkoxymethyl.

Suitable rapamycin analogues are disclosed in WO 94/09010 and WO 96/41807.

Particularly suitable rapamycin analogues include 40-O-(2-hydroxy)ethyl-rapamycin, 32-deoxo-rapamycin, 16-O-pent-2-ynyl-32-deoxo-rapamycin, 16-O-pent-2-ynyl-32-deoxo-40-O-(2-hydroxyethyl)-rapamycin, 16-O-pent-2-ynyl-32-(S)-dihydro-rapamycin and 16-O-pent-2-ynyl-32-(S)-dihydro-40-O-(2-hydroxyethyl)-rapamycin.

Other rapamycin analogues include hydroxyesters of rapamycin, such as 3-hydroxy-2-(hydroxymethyl)-2-methylpropionic acid (CCI-779). The preparation and use of hydroxyesters of rapamycin, including CCI-779, are disclosed in U.S. Pat. Nos. 5,362,718 and 6,277,983

Other rapamycin analogues include carboxylic acid esters as set out in WO 92/05179, amide esters as set out in U.S. Pat. No. 5,118,677, carbamates as set out in U.S. Pat. No. 5,118,678, fluorinated esters as set out in U.S. Pat. No. 5,100,883, acetals as set out in U.S. Pat. No. 5,151,413, silyl ethers as set out in U.S. Pat. No. 5,120,842 and arylsulfonates and sulfamates as set out in U.S. Pat. No. 5,177,203.

Other rapamycin analogues which may be used in accordance with the invention may have the methoxy group at the position 16 replaced with alkynyloxy as set out in WO 95/16691. Rapamycin analogues are also disclosed in WO 93/11130, WO 94/02136, WO 94/02385 and WO 95/14023.

A method as described herein may further comprise determining the level of autophagy in the cell. This may be carried out, for example, by assessing clearance of known autophagy substrates, measurement of LC3-positive vesicles in cells, measurement of LC3 II band by western blot or electron microscopy.

A suitable cell may be a cell from a primary cell culture or a cultured non-neuronal or neuronal cell line. Many suitable cell lines are known, for example Chinese hamster ovary, baby hamster kidney, COS cell (and in particular African green monkey kidney (COS-7)), PC12, human neuroblastoma (e.g. SK-N-SH), or human cervical carcinoma (e.g. Hela).

The cell may express an aggregation-prone polypeptide and the level of aggregates of the polypeptide, and/or the formation and clearance of aggregates, may be determined. In some embodiments, the cell may comprise a heterologous nucleic acid encoding an aggregation-prone polypeptide, for example A53T or A30P mutant forms of α-synuclein, huntingtin or GFP-tagged with expanded polyalanine repeats. An aggregation-prone polypeptide may comprise an aggregation-inducing mutation, for example a codon iteration mutation such as a polyQ or polyA insertion, or may have the non-mutant, wild-type sequence. Clearance of the encoded aggregation-prone polypeptide, either in an aggregated or a soluble Monomeric form, may be determined. Expression of the heterologous nucleic acid may be reversible, i.e. expression may be induced and repressed as required, for example by adding or removing an inducer compound. A method may comprise inducing and repressing the expression of said nucleic acid prior to contacting the mammalian cell with the test compound. Many examples of inducible and/or reversible expression system as and constructs are known in the art, including, for example the Tet-on™ expression (Clontech), in particular in combination with the pTet-tTs™ vector (Clontech).

The activity of the cAMP/EPAC/PLC pathway may be modulated, i.e. reduced or increased in vitro. Alternatively, the activity of the cAMP/EPAC/PLC pathway may be modulated, i.e. reduced or increased in a cell in vivo. The cell may, for example, be comprised in a human or non-human mammal. A cAMP/EPAC/PLC pathway antagonist may be contacted with the cell in vivo by administering the antagonist to the individual. The administration of suitable antagonist compounds is described in more detail below.

The cell may be comprised in an individual having a condition which is ameliorated by increased autophagy, for example a neurodegenerative disorder or pathogen infection, or an individual at risk of suffering from such a condition and methods of inducing autophagy as described herein may be useful for the treatment of a neurodegenerative disorder or pathogen infection or for preventing or delaying the onset of such a disorder or infection.

A individual suitable for undergoing a method of preventing or delaying the onset of a neurodegenerative disorder as described herein may have no symptoms of a neurodegenerative disorder. In some embodiments, the individual may be at risk of or susceptible to a neurodegenerative disorder, for example the individual may have one or more risk factors associated with the onset of a neurodegenerative disorder.

Neurodegenerative disorders may include protein aggregation disorders. Protein aggregation disorders (also known as protein conformation disorders or proteinopathies) are characterised by the formation of intracellular protein aggregates. Protein aggregation disorders include codon reiteration mutation disorders, α-synucleinopathies, prion disorders and tauopathies.

Codon reiteration mutation disorders include polyA expansion disorder and polyO expansion disorders. PolyA expansion disorders are characterised by a polyadenine (polyA) expansion mutation. PolyQ expansion disorders are characterised by a polyglutamine (polyQ) expansion mutation and include Huntington's disease, spinocerebellar ataxias types 1, 2, 3, 6, 7 and 17, spinobulbar muscular dystrophy and dentatorubral pallidoluysian atrophy.

α-synucleinopathies are characterised by the accumulation of Lewy bodies comprising α-synuclein, and include Parkinson's Disease, LB variant Alzheimer's disease and LB dementia.

Prion disorders are characterised by the aggregation of PrP^{Sc} and include familial, sporadic and new variant CJD, as well as veterinary disorders such as scrapies and BSE.

Tauopathies are characterised by the abnormal accumulation of tau protein, in particular hyperphosphorylated tau protein, and include sporadic frontotemporal dementia (FTD), Pick's disease and Alzheimer's disease.

A pathogen infection may be a bacterial infection, for example a streptococcal infection or a mycobacterial infection such as tuberculosis, or a viral infection, for example an Herpes Simplex Virus (HSV) or Sindbis virus infection.

Examples of suitable cAMP/EPAC/PLC pathway antagonists and mTOR inhibitors for use in the present methods are described above.

Another aspect of the invention provides a pharmaceutical composition comprising a cAMP/ERAC/PLC pathway antagonist, an mTOR inhibitor and a pharmaceutically acceptable excipient.

Suitable cAMP/EPAC/PLC pathway inhibitors are described above and include cAMP antagonists (i.e. agents which reduce the level or amount of cAMP in a cell), such as clonidine, tyramine or rilmenidine, adenyl cyclase antagonists, such as 2′5′dideoxyadenosine, Gₛα/PACAP antagonist, such as NF449, and antagonists of cAMP/EPAC/PLC pathway components such as Epac1, Rap2B and PLC-ε.

Another aspect of the invention provides a pharmaceutical composition comprising an ATP-sensitive K⁺ channel agonist, an mTOR inhibitor and a pharmaceutically acceptable excipient.

Suitable ATP-sensitive K⁺ channel agonists are described above and include minoxidil, pinacidil, cromakalim and isomers, salts, solvates, chemically protected forms, and prodrugs thereof. Other examples of ATP-sensitive K⁺ channel agonists are described in Cecchetti V et al Curr Top Med Chem. 2006; 6(10):1049-68.

Suitable mTOR inhibitors include rapamycin macrolides, such as rapamycin, as described above.

A suitable method of producing a pharmaceutical composition may comprise admixing a cAMP/EPAC/PLC pathway antagonist or an ATP-sensitive K⁺ channel agonist as described herein and an mTOR inhibitor with a pharmaceutically acceptable excipient, vehicle or carrier.

A pharmaceutically acceptable excipient, vehicle or carrier, should be non-toxic and should not interfere with the efficacy of the active ingredient. The precise nature of the carrier or other material will depend on the route of administration, which may be oral, or by injection, e.g. cutaneous, subcutaneous or intravenous.

Pharmaceutical compositions for oral administration may be in tablet, capsule, powder or liquid form. A tablet may include a solid carrier such as gelatin or an adjuvant. Liquid pharmaceutical compositions generally include a liquid carrier such as water, petroleum, animal or vegetable oils, mineral oil or synthetic oil. Physiological saline solution, dextrose or other saccharide solution or glycols such as ethylene glycol, propylene glycol or polyethylene glycol may be included.

For intravenous, cutaneous or subcutaneous injection, or injection at the site of affliction, the active ingredient will be in the form of a parenterally acceptable aqueous solution which is pyrogen-free and has suitable pH, isotonicity and stability. Those of relevant skill in the art are well able to prepare suitable solutions using, for example, isotonic vehicles such as Sodium Chloride Injection, Ringer's Injection, Lactated Ringer's Injection. Preservatives, stabilisers, buffers, antioxidants and/or other additives may be included, as required.

Examples of techniques and protocols mentioned above can be found in Remington's Pharmaceutical Sciences, 16th edition, Osol, A. (ed), 1980.

Formulations and administration regimes which are suitable for use with rapamycin macrolides such as rapamycin, and cAMP/EPAC/PLC pathway antagonists or ATP-sensitive K⁺ channel agonists as described herein, are well known in the art.

Administration is preferably in a “therapeutically effective amount”, this being sufficient to show benefit to the individual. The actual amount administered, and rate and time-course of administration, will depend on the nature and severity of what is being treated. Prescription of treatment, e.g. decisions on dosage etc, is within the responsibility of medical practitioners.

A composition may be administered alone or in combination with other treatments, either simultaneously or sequentially dependent upon the condition to be treated

Other aspects of the invention relate to methods of decreasing autophagy in a cell. This may be useful, for example, in the treatment of cancer (see, for example, Amaravadi R K et al J Clin Invest. 2007 Feb. 1; 117(2):326-336. Epub 2007 Jan. 18)

A method of decreasing autophagy in a cell may comprise;
activating or increasing the activity of the cAMP/EPAC/PLC pathway in said cell.

Activation of the cAMP/EPAC/PLC pathway reduces autophagy in the cell.

The method may further comprise determining the level of autophagy in the cell following the activation or increase. Methods of measuring autophagy in a cell are described in more detail above.

In some embodiments, the cAMP/EPAC/PLC pathway may be activated or its activity increased in a cell by increasing the amount of cytosolic 3′, 5′ cyclic adenosine monophosphate (cAMP) in said cell.

The level or amount of cystosolic cAMP may be increased by contacting the cell with a cAMP agonist, which may, for example, interact with cAMP-coupled receptors on the cell surface to modulate cAMP levels in the cytosol of the cell. A cAMP agonist may inhibit the depletion of cAMP, promote the synthesis of cAMP, promote the transport of cAMP into the cytosol, inhibit Gi/cAMP pathways and/or stimulate Gs/cAMP pathways. Suitable cAMP agonists include Gs coupled receptor agonists, Gi-coupled receptor antagonists and phophodiesterase inhibitors (see for example Cheng J and Grande. Exp Biol Med (Maywood). 2007 January; 232(1):38-51).

Examples of suitable cAMP agonists include rolipram.

Rolipram has the IUPAC name 4-[3-(Cyclopentyloxy)-4-methoxypheny]-2-pyrrolidinone (CAS 61413-54-5) and is commercially available e.g. from Sigma-Aldrich.

A cAMP agonist may for example, promote or increase the activity of a factor which stimulates, activates or increases the activity of adenylate cyclase, such as a Gs/cAMP pathway component, or inhibit or reduce the activity of a factor which reduces or inhibits the activity of adenylate cyclase, such as a Gi/cAMP pathway component.

Factors which stimulate, activate or increase the activity of adenylate cyclase include Gₛα and PACAP, which are described above. cAMP agonists may include Gₛα and/or PACAP agonists.

A cAMP agonist may, for example, promote or increase the activity of the cAMP/EPAC/PLC pathway, thereby increasing the level of cytosolic Ca²⁺, leading to the activation of calpain, Gₛα and PACAP and increased adenylate cyclase activity.

The cAMP/EPAC/PLC pathway may be activated in the cell by increasing or promoting the activity of one or more components of the pathway, such as Epac1, Rap2B and PLC-ε. cAMP agonists may thus include Epac1, Rap2B and/or PLC-ε agonists.

A cAMP agonist may act directly on adenylate cyclase to promote or increase its activity. cAMP agonists may include adenylate cyclase agonists, such as forskolin.

Forskolin has the IUPAC name 7β-Acetoxy-8,13-epoxy-1α,6β,9α-trihydroxylabd-14-en-11-one (CAS 66575-29-9) and is commercially available e.g. from Sigma-Aldrich.

A cAMP agonist may, for example, reduce or inhibit the activity of an ATP-sensitive K⁺ channel, thereby increasing the level of cytosolic Ca²⁺. cAMP agonists thus include ATP-sensitive K⁺ channel antagonists such as tetraethylammonium (CAS 67533-12-4), apamin (CAS 24345-16-2), charybdotoxin (CAS 95751-30-7), gliquidone (3-cyclohexyl-1-[4-[2-(7-methoxy-4,4-dimethyl-1,3-dioxo-isoquinolin-2-yl)ethyl]phenyl]sulfonyl-urea: CAS 33342-05-1, tolezamide (3-azepan-1-yl-1-(4-methylphenyl)sulfonyl-urea: CAS 1156-19-0), tolbutamide (1-Butyl-3-(4-methylphenylsulfonyl)urea: CAS 64-77-7) and chloropropamide (1-(4-chlorophenyl)sulfonyl-3-propyl-urea: CAS 94-20-2).

Autophagy may be reduced as described herein in vitro.

Alternatively, the activity of the autophagy may be reduced in a cell in vivo i.e. the cell may be comprised in a human or non-human mammal. A cAMP agonist or other compound described above may be contacted with the cell in vivo by administering the compound to the individual. The administration of suitable compounds is described in more detail below.

The cell may be comprised in an individual having a condition which is ameliorated by decreased autophagy, for example cancer, and methods of reducing autophagy as described herein may be useful for the treatment of cancer or for preventing or delaying the onset of cancer. Other aspects of the invention provide a method of treating cancer comprising administering a cAMP agonist as described herein to an individual in need thereof, a cAMP agonist as described herein for treating cancer and the use of a cAMP agonist as described herein in the manufacture of a medicament for use in the treatment of cancer.

Other aspects of the invention relate to methods of identifying and/or obtaining compounds which induce autophagy in a cell and which may therefore be useful in the treatment of a neurodegenerative disorder or pathogen infection.

A method of identifying and/or obtaining a compound which induces autophagy in a cell may comprise,
determining the ability of a test compound to reduce the activity of the cAMP/EPAC/PLC pathway in a cell.

In some preferred embodiments, the ability of a test compound to reduce the activity of the cAMP/EPAC/PLC pathway in a cell is determined by determining the effect of the test compound on the level of cAMP in the cell. A method of identifying and/or obtaining a compound which induces autophagy in a cell may comprise,
determining the ability of a test compound to reduce the level or amount of cAMP in a cell.

The ability of a test compound to reduce the level or amount of cAMP in a cell may be determined by contacting a cell with the test compound and measuring the change in the amount of cAMP in the cytosol of the cell in the presence relate to the absence of the test compound. A decrease in cAMP in the presence of the test compound relative to controls is indicative that the test compound induces autophagy in a cell.

The level or amount of cAMP in a cell may be measured by conventional techniques as described above.

Compounds may reduce the level or amount of cAMP in a cell through an antagonistic effect on adenylate cyclase. A method of identifying and/or obtaining a compound which induces autophagy in a cell may comprise,
contacting an adenylate cyclase protein with a test compound and determining the activity of the adenylate cyclase protein in the presence relative to the absence of the test compound,
wherein an decrease in adenylate cyclase activity in the presence, relative to the absence of test compound is indicative that the test compound induces autophagy in a cell.

Compounds may reduce the level or amount of cAMP in a cell through an antagonistic effect on Gₛα, and/or PACAP. A method of identifying and/or obtaining a compound which induces autophagy in a cell may comprise,
contacting an Gₛα and/or a PACAP protein with a test compound and
determining the activity of the protein in the presence relative to the absence of the test compound,
wherein an decrease in activity in the presence, relative to the absence of test compound is indicative that the test compound induces autophagy in a cell.

Compounds may reduce the level or amount of cAMP in a cell through an antagonistic effect on one or more components of the cAMP/EPAC/PLC pathway, such as Epac1, Rap2B and PLC-ε. A method of identifying and/or obtaining a compound which induces autophagy in a cell may comprise,
contacting a component of the cAMP/EPAC/PLC pathway with a test compound and
determining the activity of the component in the presence relative to the absence of the test compound,
wherein an decrease in component activity in the presence, relative to the absence of test compound is indicative that the test compound induces autophagy in a cell.

A component of the cAMP/EPAC/PLC pathway may be selected from the group consisting of Epac1, Rap2B and PLC-ε.

Compounds may reduce the level or amount of cAMP in a cell through an agonistic effect on ATP-sensitive K⁺ channels. A method of identifying and/or obtaining a compound which induces autophagy in a cell may comprise,
contacting an ATP-sensitive K⁺ channel protein with a test compound and
determining the activity of the ATP-sensitive K⁺ channel protein in the presence relative to the absence of the test compound,
wherein an increase in channel activity in the presence, relative to the absence of test compound is indicative that the test compound induces autophagy in a cell.

A test compound which is identified using one or more of the above methods as being a compound which induces autophagy in a cell may be useful in the treatment of a neurodegenerative disorder or pathogen infection.

A suitable target protein for use in a screening method described above, for example an adenylate cyclase protein, Gₛα protein, PACAP protein, ATP-sensitive K⁺ channel protein, or component of the cAMP/EPAC/PLC pathway, may be a protein from any higher eukaryote, preferably a mammalian species, such as mouse or human, or may be fragment or variant of the sequence of any one of these.

A fragment or variant of a target protein as described herein may have a sequence which differs from the sequence of the reference target protein by the addition, deletion, substitution and/or insertion of one or more amino acids, provided activity is retained. Suitable target proteins and the database entries of reference sequences for these proteins are set out above.

A polypeptide which is a variant of a reference sequence may comprise an amino acid sequence which shares greater than about 30% sequence identity with the reference sequence, greater than about 40%, greater than about 45%, greater than about 55%, greater than about 65%, greater than about 70%, greater than about 80%, greater than about 90% or greater than about 95%. The sequence may share greater than about 30% similarity with the reference sequence, greater than about 40% similarity, greater than about 50% similarity, greater than about 60% similarity, greater than about 70% similarity, greater than about 80% similarity or greater than about 90% similarity.

Sequence similarity and identity are commonly defined with reference to the algorithm GAP (Genetics Computer Group, Madison, Wis.). GAP uses the Needleman and Wunsch algorithm to align two complete sequences that maximizes the number of matches and minimizes the number of gaps. Generally, default parameters are used, with a gap creation penalty=12 and gap extension penalty=4. Use of GAP may be preferred but other algorithms may be used, e.g. BLAST (which uses the method of Altschul et al. (1990) J. Mol. Biol. 215: 405-410), FASTA (which uses the method of Pearson and Lipman (1988) PNAS USA 85: 2444-2448), or the Smith-Waterman algorithm (Smith and Waterman (1981) J. Mol Biol. 147: 195-197), or the TBLASTN program, of Altschul et al. (1990) supra, generally employing default parameters. In particular, the psi-Blast algorithm (Nucl. Acids Res. (1997) 25 3389-3402) may be used. Sequence identity and similarity may also be determined using Genomequest™ software (Gene-IT, Worcester Mass. USA).

Sequence comparisons are preferably made over the full-length of the relevant sequence described herein.

Similarity allows for “conservative variation”, i.e. substitution of one hydrophobic residue such as isoleucine, valine, leucine or methionine for another, or the substitution of one polar residue for another, such as arginine for lysine, glutamic for aspartic acid, or glutamine for asparagine.

Determining the activity of a target protein may include detecting the presence of activity, detecting the presence of activity above a threshold value and/or measuring the level of activity.

As described above, polypeptide fragments which retain all or part of the activity of the full-length protein may be generated and used in the methods described herein, whether in vitro or in vivo. Suitable ways of generating fragments include recombinant techniques and chemical synthesis techniques which are well known in the art.

A fragment of a full-length sequence may consist of fewer amino acids than the full-length sequence. For example a fragment may consist of at least 10, at least 20, at least 30, at least 40, at least 50, at least 60, at least 70, at least 80, at least 90 or at least 100 amino acids of the full length sequence but 800 or less, 700 or less, 600 or less, 500 or less, 250 or less, 200 or less, 150 or less, or 125 or less amino acids of the full length sequence.

Screening methods described herein may be in vivo cell-based methods, or in vitro non-cell-based methods. The precise format for performing methods of the invention may be varied by those of skill in the art using routine skill and knowledge.

A test compound may be contacted with a cell and the level or amount of autophagy in said cell determined. An increase in autophagy in the presence relative to the absence of test compound is indicative that the compound may be useful in the treatment of a neurodegenerative disorder or a pathogen infection.

A method may comprise identifying the test compound as useful in the treatment of a neurodegenerative disorder or a pathogenic infection.

Compounds which may be screened using the methods described herein may be natural or synthetic chemical compounds used in drug screening programmes. Extracts of plants, microbes or other organisms which contain several characterised or uncharacterised components may also be used.

Combinatorial library technology provides an efficient way of testing a potentially vast number of different compounds for ability to modulate an interaction. Such libraries and their use are known in the art, for all manner of natural products, small molecules and peptides, among others. The use of peptide libraries may be preferred in certain circumstances.

The amount of test compound or compound which may be added to a method of the invention will normally be determined by trial and error depending upon the type of compound used. Typically, from about 0.001 nM to 1 mM or more of putative inhibitor compound may be used, for example from 0.01 nM to 100 μM, e.g. 0.1 to 50 μM, such as about 10 μM.

Suitable test compounds for screening include known modulators of cAMP levels, compounds such as antagonists of adenylate cyclase, Epac1, Rap2B, PLC-ε, Gₛα and/or PACAP which are identified herein as modulators of cAMP levels, and analogues and mimetics of these, for example compounds produced using rational drug design to provide candidate compounds with particular molecular shape, size and charge characteristics suitable for inducing autophagy in mammalian cells.

A method may comprise identifying the test compound as an autophagy inducer. Such a compound may for example be useful in the treatment of a neurodegenerative disorder or a pathogenic infection.

A test compound identified using one or more initial screens as having ability to increase or induce autophagy in a cell may be assessed further using one or more secondary screens. A secondary screen may, for example, involve testing for a biological function such as clearance of protein aggregates and amelioration of symptoms of neurodegenerative disorder or a pathogenic infection in animal models.

The test compound may be isolated and/or purified or alternatively, it may be synthesised using conventional techniques of recombinant expression or chemical synthesis. Furthermore, it may be manufactured and/or used in preparation, i.e. manufacture or formulation, of a composition such as a medicament, pharmaceutical composition or drug. These may be administered to individuals for the treatment of disease conditions such as a neurodegenerative disorder or a pathogenic infection, as described below, or for preventing or delaying the onset of such a condition. Methods described herein may thus comprise formulating the test compound in a pharmaceutical composition with a pharmaceutically acceptable excipient, vehicle or carrier for therapeutic application, as discussed further below.

Following identification of a compound which induces autophagy in a cell, a method may further comprise modifying the compound to optimise its pharmaceutical properties.

The modification of a ‘lead’ compound identified as biologically active is a known approach to the development of pharmaceuticals and may be desirable where the active compound is difficult or expensive to synthesise or where it is unsuitable for a particular method of administration, e.g. peptides are not well suited as active agents for oral compositions as they tend to be quickly degraded by proteases in the alimentary canal. Modification of a known active compound (for example, to produce a mimetic) may be used to avoid randomly screening large number of molecules for a target property.

Modification of a ‘lead’ compound to optimise its pharmaceutical properties commonly comprises several steps. Firstly, the particular parts of the compound that are critical and/or important in determining the target property are determined. In the case of a peptide, this can be done by systematically varying the amino acid residues in the peptide, e.g. by substituting each residue in turn. These parts or residues constituting the active region of the compound are known as its “pharmacophore”.

Once the pharmacophore has been found, its structure is modelled to according its physical properties, e.g. stereochemistry, bonding, size and/or charge, using data from a range of sources, e.g. spectroscopic techniques, X-ray diffraction data and NMR.

Computational analysis, similarity mapping (which models the charge and/or volume of a pharmacophore, rather than the bonding between atoms) and other techniques can be used in this modelling process.

In a variant of this approach, the three-dimensional structure of the compound which induces autophagy is modelled. This can be especially useful where the compound changes conformation, allowing the model to take account of this in the optimisation of the lead compound.

A template molecule is then selected, onto which chemical groups that mimic the pharmacophore can be grafted. The template molecule and the chemical groups grafted on to it can conveniently be selected so that the modified compound is easy to synthesise, is likely to be pharmacologically acceptable, and does not degrade in vivo, while retaining the biological activity of the lead compound. The modified compounds found by this approach can then be screened to see whether they have the target property, or to what extent they exhibit it. Modified compounds include mimetics of the lead compound.

Further optimisation or modification can then be carried out to arrive at one or more final compounds for in vivo or clinical testing.

As described above, a compound identified and/or obtained using the present methods may be formulated into a pharmaceutical composition as described above.

Various further aspects and embodiments of the present invention will be apparent to those skilled in the art in view of the present disclosure. All documents mentioned in this specification are incorporated herein by reference in their entirety.

Unless context dictates otherwise, the descriptions and definitions of the features set out above are not limited to any particular aspect or embodiment of the invention and apply equally to all aspects and embodiments which are described.

“and/or” where used herein is to be taken as specific disclosure of each of the two specified features or components with or without the other. For example “A and/or B” is to be taken as specific disclosure of each of (i) A, (ii) B and (iii) A and B, just as if each is set out individually herein.

Certain aspects and embodiments of the invention will now be illustrated by way of example and with reference to the figures described above and tables described below.

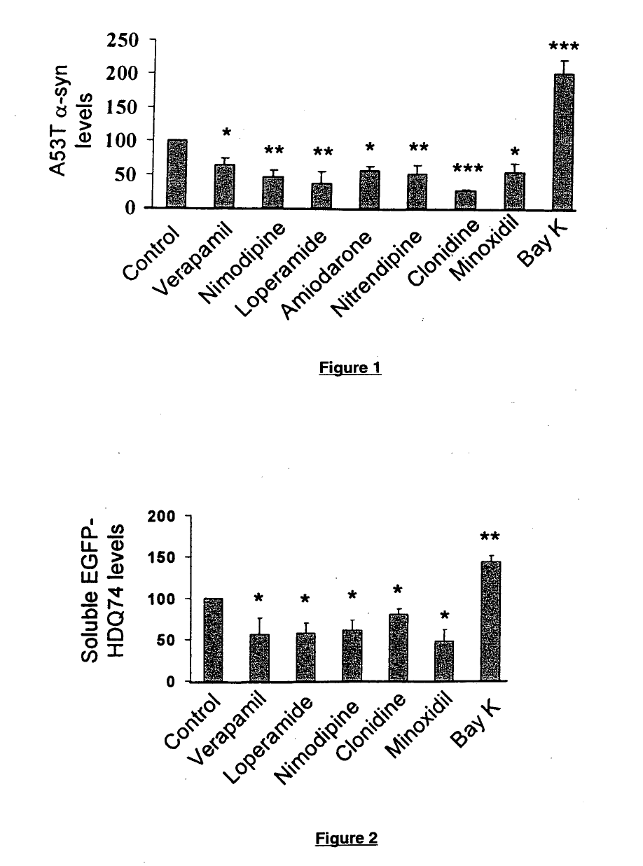

FIG. 1 shows densitometric analysis relative to actin of clearance in stable inducible PC12 cell line expressing A53T α-synuclein. Transgene expression was induced with doxycycline for 48 h, then switched off (by removing doxycycline) for 24 h and treated with or without verapamil, nimodipine, loperamide, amiodarone, nitrendipine clonidine, minoxidil or (±)-Bay K8644 (all 1 μM). DMSO was control. A53T α-synuclein was detected with anti-HA antibody.

FIG. 2 shows densitometric analysis relative to actin of clearance in stable inducible PC12 cell line expressing EGFP-HDQ74. Transgene expression was induced with doxycycline for 8 h, then switched off (by removing doxycycline) for 96 h and treated with or without verapamil, loperamide, nimodipine, clonidine, minoxidil or (±)-Bay K8644 (all 1 μM). DMSO was control.

FIG. 3 shows the proportion of EGFP-positive SK-N-SH cells with aggregates or cell death expressed as odds ratios with the control was taken as 1. SK-N-SH cells transfected with EGFP-HDQ74 construct for 4 h were treated with or without verapamil, loperamide, nimodipine, clonidine, minoxidil or (±)-Bay K8644 (all 1 μM) for 48 h post-transfection. DMSO was control. The proportions of EGFP-positive cells with aggregates or cell death were expressed as odds ratios and the control was taken as 1. Typical control values were 40% for aggregation and 20% for cell death. Odds ratios are used as summary statistics for many experiments to allow comparisons across multiple independent experiments (see methods).

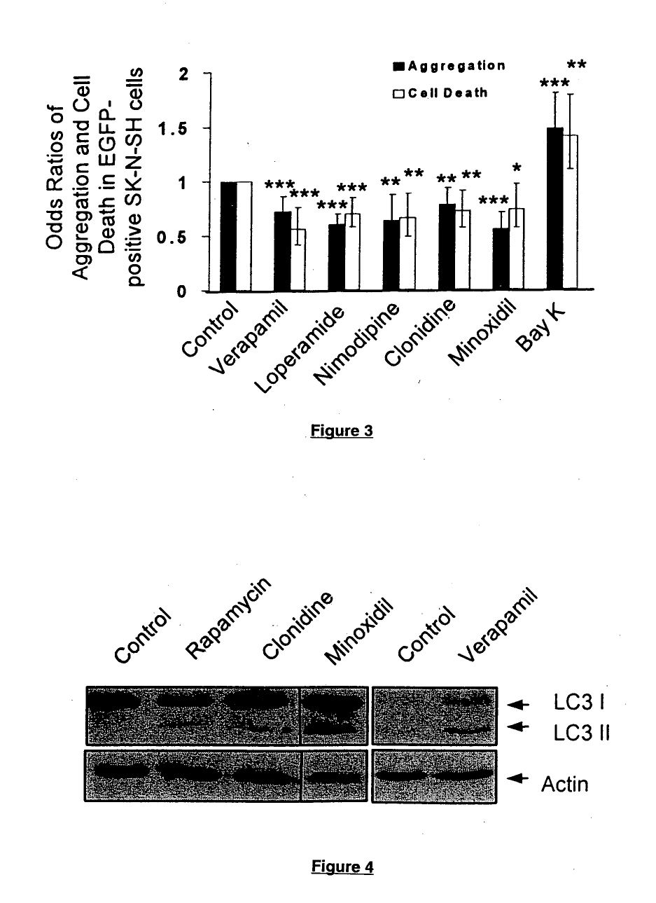

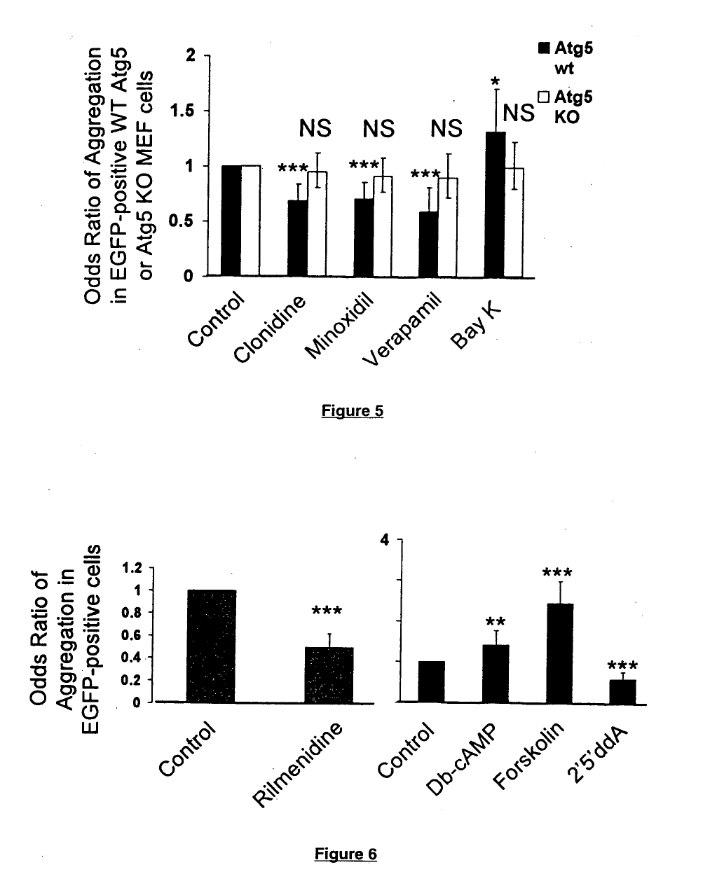

FIG. 4 shows the detection of endogenous LC3-II levels using anti-LC3 antibody for PC12 cells treated with or without 1 μM clonidine, 1 μM minoxidil and 1 μM verapamil for 24 h. Rapmaycin (0.2 μM) was used as a positive control.

FIG. 5 shows the proportions of EGFP-positive cells with EGFP-HDQ74 aggregates in wild type (WT) and knockout (KO) Atg5 mouse embryonic fibroblasts (MEFs), transfected with EGFP-HDQ74 for 4 h and then treated for next 48 h with or without 1 μM clonidine, 1 μM minoxidil, 1 μM verapamil, or 1 μM (±)-Bay K8644, expressed as odds ratio. Note that all data are from the same Atg5-deficient (KO) cells had increased EGFP-HDQ74 aggregates compared to wild-type Atg5 cells due to impairment of autophagy. Typical control values were 45% and 60% for aggregation in Atg5 wt and KO cells respectively. p<0.0001. ***, p<0.001; **, p<0.01; *, p<0.05; NS, Non-significant.

FIG. 6 shows the proportions of EGFP-positive cells with EGFP-HDQ74 aggregates in SK-N-SH (for rilmenidine) and COS-7 (for db-cAMP, forskolin, 2′5′ ddA), treated with or without 1 μM rilmenidine, 1 mM dibutyl-cAMP (db-cAMP), 24 μM forskolin or 500 μM 2′5′ dideoxyadenosine (2′5′ ddA), for 48 hr post-transfection.

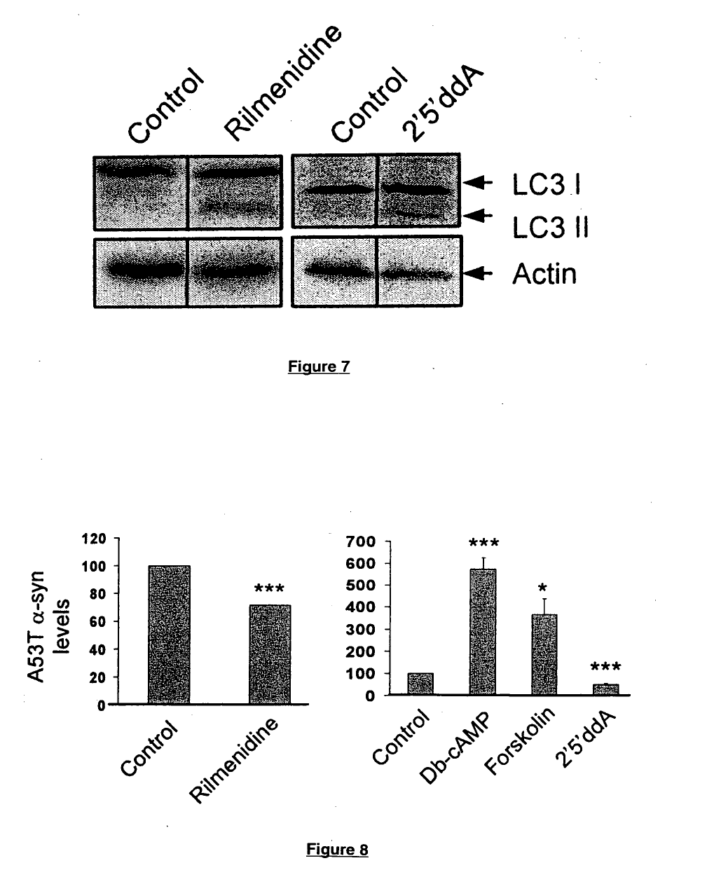

FIG. 7 shows endogenous LC3-II levels detected using anti-LC3 antibodies in PC12 cells treated with or without 1 μM rilmenidine or 500 μM 2′5′ dideoxyadenosine (2′5′ ddA) for 24 h.

FIG. 8 shows densitometric analysis relative to actin of A53T α-synuclein clearance in stable PC12 cells as in FIG. 1A, treated with water (control) or 1 μM rilmenidine, 500 μM 2′5′ dideoxyadenosine (2′5′ ddA), 24 μM forskolin or 1 mM dibutyl-cAMP (db-cAMP) for 24 h.

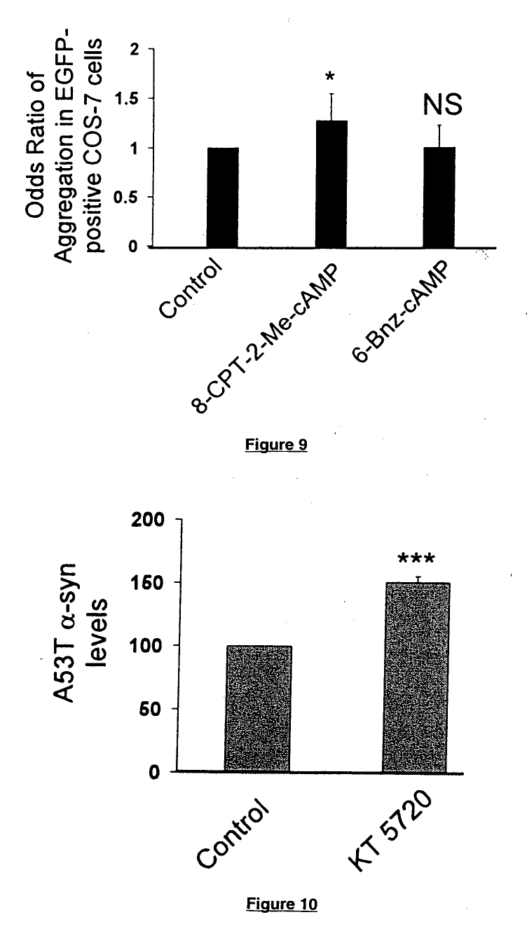

FIG. 9 shows the proportions of EGFP-positive cells with EGFP-HDQ74 aggregates in COS-7, treated with or without 10 μM 8-CPT-2Me-cAMP or 10 μM 6-Bnz-cAMP for 48 h post-transfection.

FIG. 10 shows densitometry analysis of A53T α-synuclein clearance in stable PC12 cells, treated with DMSO (control) or 1 μM KT5720 for 24 h, relative to actin.

FIG. 11 shows the proportion of COS-7 cells with aggregates following transfection with EGFP-HDQ74 construct along with empty vector (pcDNA3.1), dominant-negative Rap2B (Rap2BS17N) or wild-type PLC-ε at a ratio of 1:3. The proportions of EGFP-positive cells with aggregates were assessed after 48 h.

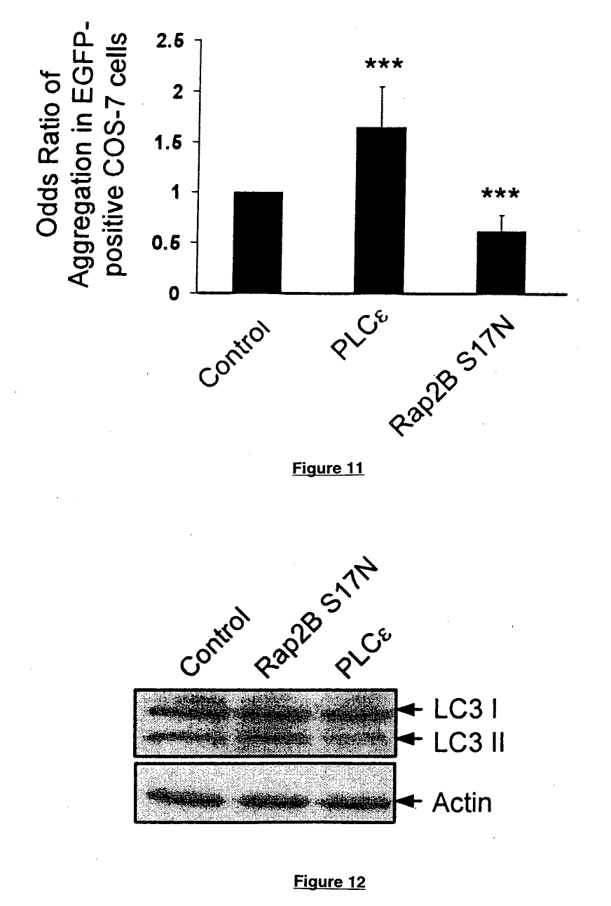

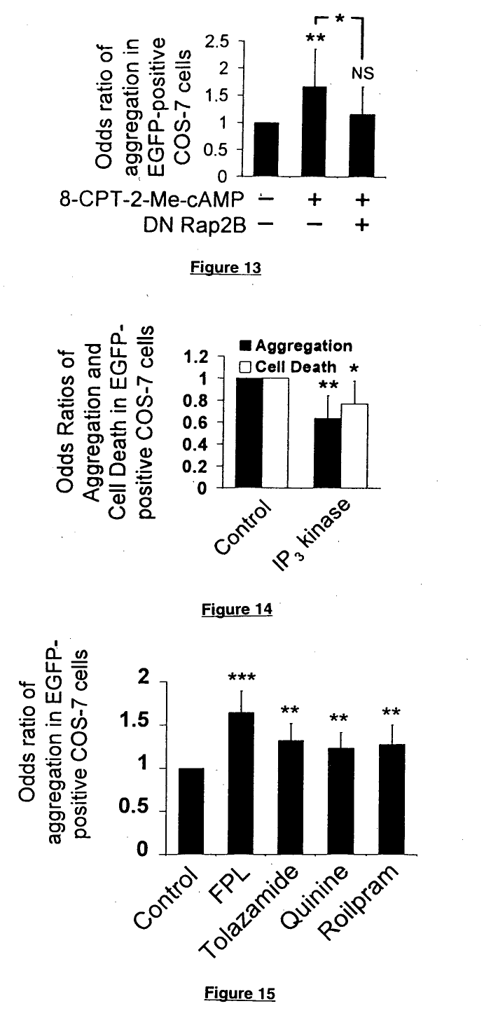

FIG. 12 shows endogenous LC3-11 levels detected using anti-LC3 antibodies in COS-7 cells transfected with a dominant-negative Rap2B (Rap2BS17N) or wild-type PLC-ε after 48 h expression.

FIG. 13 shows the proportion of EGFP-positive COS-7 cells with aggregates, transfected with EGFP-HDQ74 along with empty vector (pcDNA3.1) or dominant-negative Rap2B (1:3 ratio) for 4 h, were treated with or without 10 μM 8-CPT-2Me-cAMP for 48 h. Error bars: 95% confidence interval.

FIG. 14 shows the proportion of COS-7 cells with EGFP-HDQ74 aggregates and cell death 48 h post-transfection following transfection with EGFP-HDQ74 and pCNDA3.1 or IP₃ kinase (1:3 ratio) for 4 h. ***, p<0.001; **, p<0.01; *, p<0.05.

FIG. 15 shows the proportion of COS-7 cells with EGFP-HDQ74 aggregates 48 hours post transfection after treatment with DMSO (control), 10 μM, 10 μM tolazamide, 10 μM quinine sulphate or 10 μM rolipram.

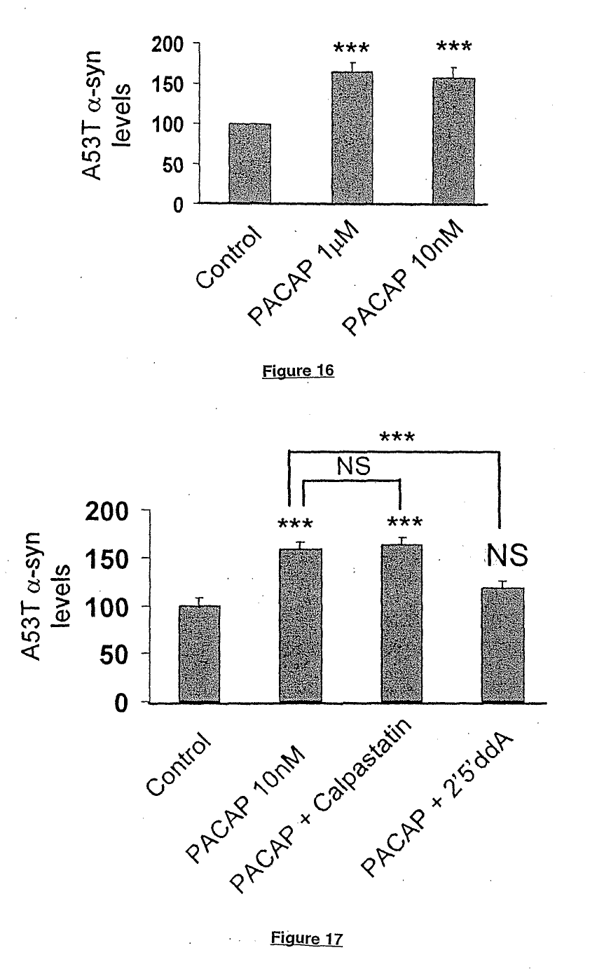

FIG. 16 shows densitometric analysis relative to actin of A53T α-synuclein clearance, treated with or without 1 μM or 10 nM pituitary adenylate-cyclase activating polypeptide (PACAP).

FIG. 17 shows densitometric analysis relative to actin of A53T α-synuclein clearance, treated with 10 nM PACAP with or without 10μ,M calpastatin or 500 μM 2′5′ ddA for 24 h.

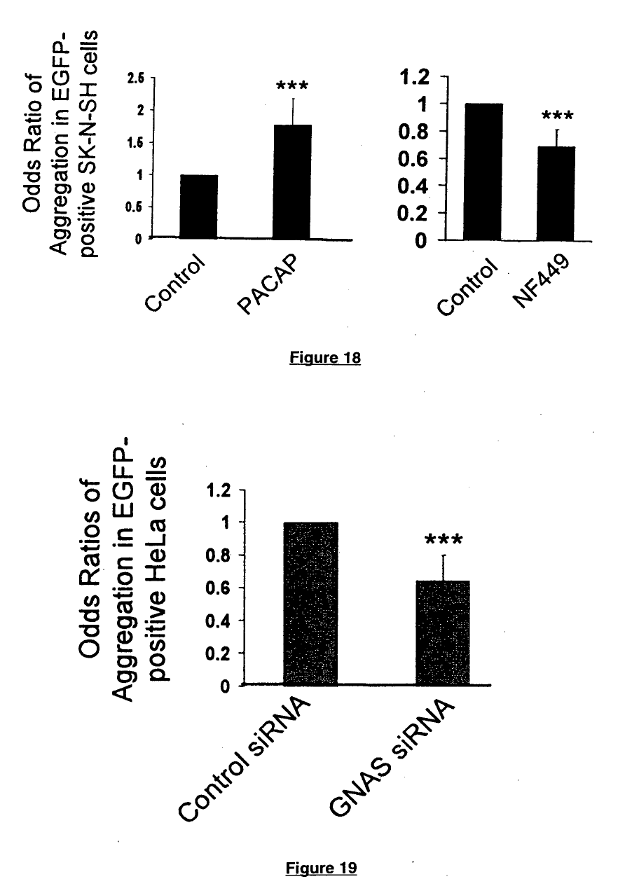

FIG. 18 shows the proportion of EGFP-positive cells with EGFP-HDQ74 aggregates in SK-N-SH cells as in FIG. 1C, treated for 48 h with 1 μM PACAP or 200 μM NF449.

FIG. 19 shows the proportion of HeLa cells with EGFP-HDQ74 aggregates in control and GNAS knockdown cells, expressed as odds ratio, following transfection with control siRNA or an siRNA smartpool against Gₛα (GNAS) and EGFP-HDQ74 for 72 h.

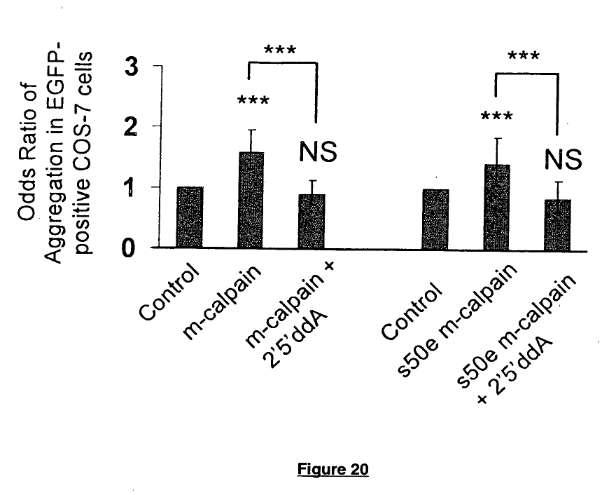

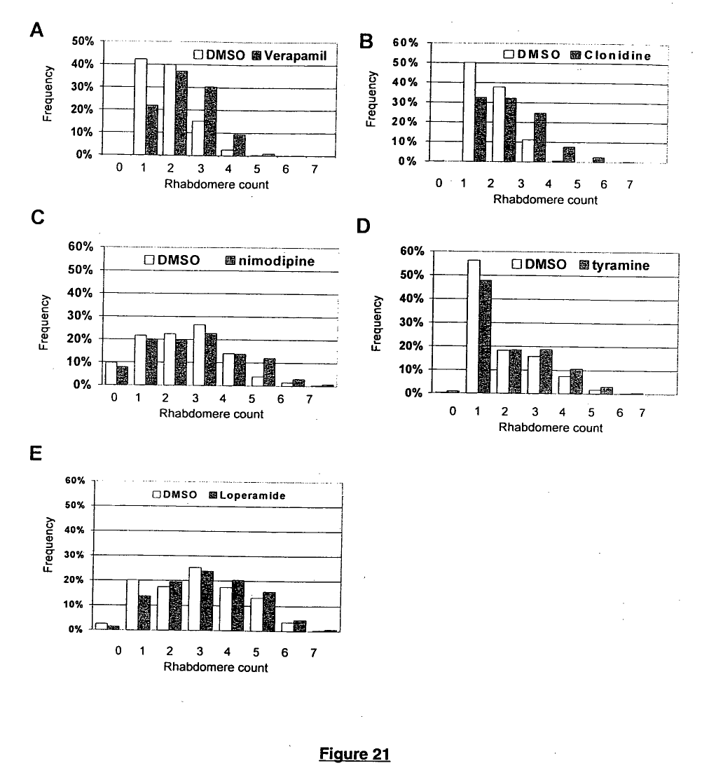

FIG. 20 shows the proportion of COS-7 cells with EGFP-HDQ74 aggregates following transfection with either pcDNA3:1, human m-calpain or constituvely active human m-calpain and EGFP-HDQ74 in a 3:1 ratio for 4 h then treated with or without 500 μM 2′5′ ddA for 48 h.

FIG. 21 shows that L-type Ca²⁺ channel antagonists or cAMP antagonists rescue Huntington phenotypes in HD flies. Flies treated with either 1.87 mM verapamil hydrochloride, 500 μM clonidine, 500 μM, nimodipine, 500 μM tyramine or 500 μM loperamide show a shift in the distribution of the number of rhabdomeres compared to flies treated with DMSO (control) alone (2 days after eclosion). Rhabdomere counts from all 3 independent experiments are included. n=945 ommatidia (DMSO) and n=930 ommatidia (verapamil hydrochloride treatment). Verapamil also protected with 1.25 mM and 2.5 mM. Student's t-test p=0.038 using mean from each independent experiments. Mann-Whitney values for each experiment. p<0.0001. ***, p<0.001; **, p<0.01; *, p<0.05; NS, Non-significant.

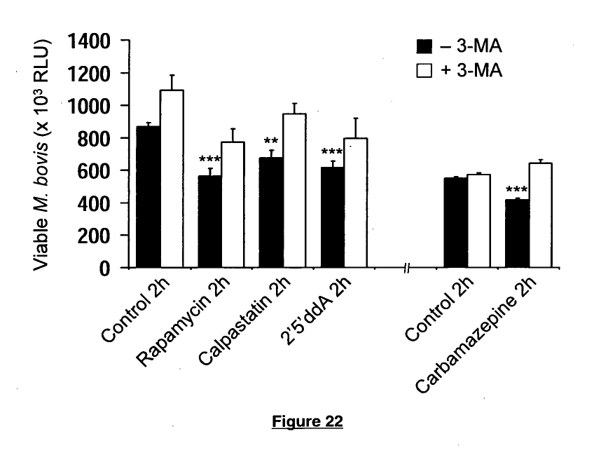

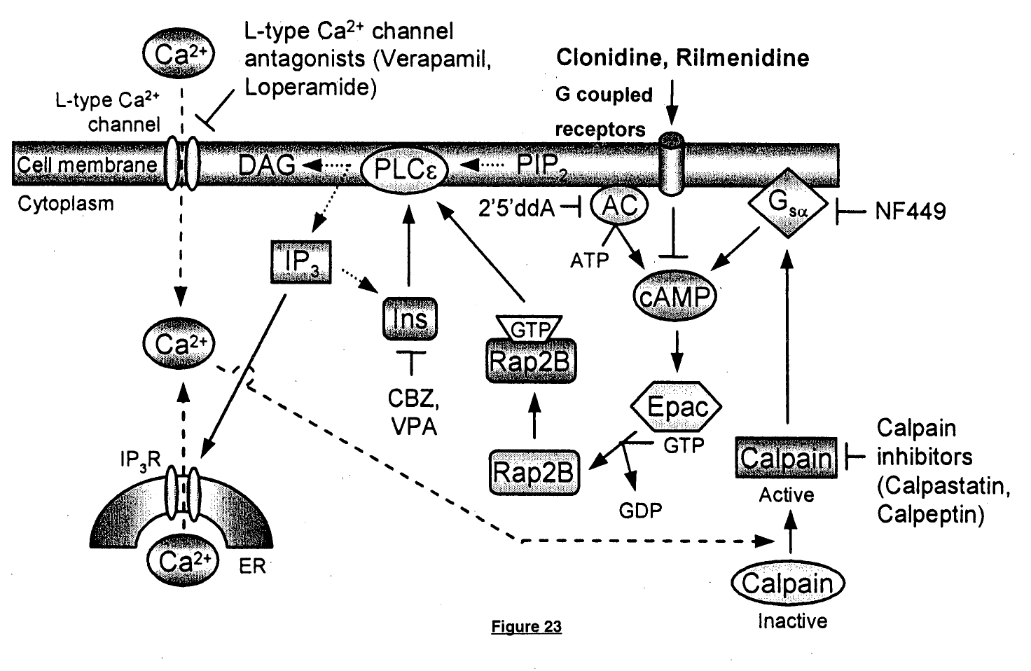

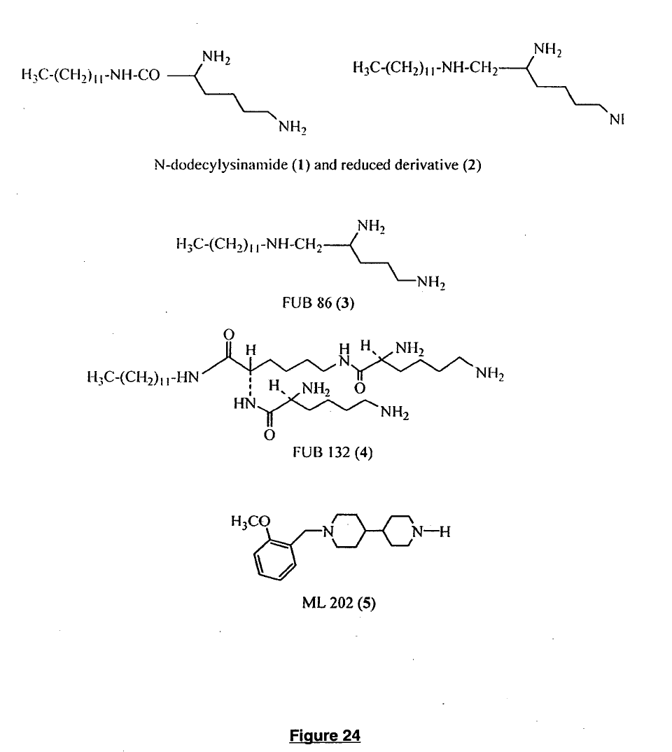

FIG. 22 shows the effect of inducing mTOR-independent autophagy on intracellular mycobacterial replication. Macrophages from healthy volunteers were incubated for 1 h with M. bovis BCGlux, washed to remove non-internalised mycobacteria, and incubated for 2 h with 0.2 μM rapamycin, 10 μM calpastatin, 500 μM 2′5′ ddA or 50 μM carbamazepine in the presence or absence of 10 mM 3MA. Carbamazepine was studied in different experiments to the other compounds. Cell associated luminescence (correlating with viable mycobacteria) was determined in triplicate samples. Results are representative of 3 separate experiments.

FIG. 23 shows a schematic representation of the novel mTOR-independent mammalian autophagy pathway, showing the potential therapeutic targets for neurodegenerative disorders and pathogen infection.

FIG. 24 shows examples of cAMP antagonists.

FIG. 25 shows further examples of cAMP antagonists.

### EXPERIMENTS

### Materials and Methods

### Clearance of Mutant Huntingtin and A53T α-Synuclein

Stable inducible PC12 cell lines expressing EGFP-HDQ74, or A53T α-synuclein mutant were induced with 1 μg/ml doxycycline (Sigma) for 8 h and 48 h respectively. Transgene expression was switched off by removing doxycycline from medium (1, 2). Cells were treated with or without compounds for time-points as indicated in experiments. If transgene levels are followed at various times after switching off expression after an initial induction period, the effect of different drugs on its clearance can be assessed, as its expression decays when synthesis is stopped. Compounds were replenished every 24 h for EGFP-HDQ74 clearance. Clearance of soluble mutant huntingtin or A53T α-synuclein was detected with anti-EGFP or anti-HA antibody respectively.

### Quantification of Aggregate Formation, Cell Death and EGFP-LC3 Vesicles

Approximately 200 EGFP-positive cells were counted for the proportion of cells with EGFP-HDQ74 aggregates, as described previously (3). Nuclei were stained with DAPI and those showing apoptotic morphology were considered abnormal. Experiments were done in triplicate, with the experimenter blinded to the treatment. Approximately 100 EGFP-positive cells were counted for the proportion of cells with >5 EGFP-LC3 vesicles, as described previously (4).

### Statistical Analysis

Pooled estimates for the changes in aggregate formation, cell death or EGFP-LC3 vesicles, resulting from perturbations assessed in multiple experiments, were calculated as odds ratios with 95% confidence intervals. Odds ratios and p values were determined by unconditional logistical regression analysis, using the general log-linear analysis option of SPSS 9 software (SPSS, Chicago). Densitometry analysis on the immunoblots was done by Scion Image Beta 4.02 software (Scion Corporation) from three independent experiments (n=3). Significance for the clearance of mutant proteins was determined by factorial ANOVA test using STATVIEW software, version 4.53 (Abacus Concepts). The control condition was set to 100% and the error bars denote standard error of mean. ***, p<0.001; **, p<0.01; *, p<0.05; NS, Non-significant.

### Compounds

Cells were treated with various compounds for various time-points as stated under different experimental conditions including 1 μm verapamil hydrochloride, 1 μM loperamide hydrochloride, 1 μM nimodipine, 1 μM nitrendipine, 1 μM amiodarone hydrochloride, 1 μM, clonidine hydrochloride, 1 μM rilmenidine, 1 μM (±)-Bay K8644, 1 μM KT5720, 1 μM minoxidil, 1 μM (S)-(−)-Bay K 8644, 1 μM (R)-(+)-Bay K 8644, 2.5 μM thapsigargin, 0.2 μM rapamycin, 400 nM bafilomycin A1, 10 mM 3-methyladenine (3-MA) and 10 μM lactacystin (all from Sigma). 10 μM calpastatin, 50 μM ALLM, 50 μM calpeptin, 20 μM caspase inhibitor I, 10 μM ionomycin, 24 μM Forskolin, 10 nM/1 μM PACAP 38, Ovine and 500 μM 2′S′dideoxyadenosine (2′S′ddA) (all from Calbiochem). 1 μm/10 μm N⁶Benzoyl-cAMP (6-Bnz-cAMP), 1 μM/10 μM 8-(4-Chlorophenylthio)-2′-0-methyl-cAMP (8-CPT-2-Me-cAMP and 1 Mm N⁶,2-O-Dibutyryl-cAMP (db-cAMP) (all from Biolog).

### Mammalian Cell Lines, Culture and Transfection

African green monkey kidney cells (COS-7), human neuroblastoma cells (SK-N-SH), human cervical carcinoma cells (HeLa), HeLa cells stably expressing EGFP-LC3 (9), Atg5 wild-type and knock-out mouse embryonic fibroblasts (6) (MEFs) were maintained in Dulbecco's Modified Eagle Medium (DMEM, Sigma) supplemented with 10% Fetal Bovine Serum (FBS, Sigma), 100 U/ml Penicillin/Streptomycin and 2 mM L-Glutamine (Sigma) at 37° C., 5% Carbon dioxide (CO₂). Cells were plated in six-well dishes at a density of 1×10⁵ cells per well for 24 h and transfected with pEGFP-HDQ74 (1.5 μg/well of 6-wells plate) using LipofectAMINE reagent for COS-7 cells and LipofectAMINE PLUS reagent for SK-N-SH cells using manufacturer's protocol (Invitrogen). The transfection mixture was replaced after 4 h incubation at 37° C. by various compounds. Transfected cells were fixed with 4% paraformaldehyde (Sigma) after 48 h and mounted in 4′,6-diamidino-2-phenylindole (DAPI, 3 mg/ml, Sigma) over coverslips on glass slides and analysed for aggregation and cell death. For immunoblotting, COS-7 cells were plated at a density of 3×10⁵ cells per well and treated for 24 h. HeLa cells stably expressing Ub^{G76V}-GFP reporter (10) (kind gift from N. P. Dantuma) were grown in the same media used for COS-7 cells supplemented with 0.5 mg/ml G418.

Inducible PC12 stable cell lines expressing EGFP-tagged exon 1 of HD gene (EGFP-HDQ74) (11) and HA-tagged A53T α-synuclein mutant(1), previously characterised, were maintained at 75 μg/ml hygromycin B (Calbiochem) in standard DMEM with 10% horse serum (Sigma), 5% FBS, 100 U/ml penicillin/streptomycin, 2 mM L-glutamine, and 100 μg/ml G418 (GIBCO) at 37° C., 10% CO₂.

### Drosophila Analysis

Fly culture and crosses were carried out at 25° C. and at 70% humidity, using Instant Fly Food (Philip Harris, Ashby de la Zouch, UK) unless otherwise stated. Flies were raised with a 12 h light: 12 h dark cycle. Aliquots of 0.5 M verapamil hydrochloride (Sigma, Poole, UK) in DMSO, or DMSO alone were added to the water that was used to prepare the instant fly food.

Virgin female flies of the genotype y w; gmr-httNterm(1-171)Q120 (gmrQ120) (12) were allowed to mate with male flies from an isogenised w¹¹¹⁸ stock (13) in food vials for 48 h. Flies were then transferred to vials containing instant fly food containing either verapamil hydrochloride in DMSO or DMSO alone. Progeny were collected 0-4 h after eclosion, kept on food of the same composition as they had been reared on, and scored for photoreceptor degeneration using the pseudopupil technique (14) two days after eclosion.

We analysed toluidine blue-stained plastic sections of gmr-httQ120 Drosophila eyes and characterized the rhabdomere loss in detail. Consistent with a previous report (12), we observed loss of rhabdomeres followed by degeneration of the eyes, which manifested as structural disorganization but resulted in only subtle and low levels of photoreceptor loss (15). No loss of rhabdomeres is seen in either wild-type flies, or transgenic flies expressing otherwise identical huntingtin transgenes with 23 glutamines. Since we have observed some variability in the eye disorganization in Q120 flies and since it is difficult to quantify structural changes, we have used the pseudopupil technique as a quantifiable read-out. The loss of visible rhabdomeres in this model preceded photoreceptor death/loss assessed by toludine-blue staining of plastic sections and is a progressive degenerative phenotype seen only in flies expressing the mutant transgene (but not the wild-type transgene) and was not present at eclosion (12).

For pseudopupil analysis, heads were removed from adult male flies and mounted on microscope slides using nail polish. The ommatidia were analyzed using a 100× objective and bright field optics with bright illumination, with the observer blinded to the identity of the flies. Rhabdomere counts were carried out by analyzing 15 ommatidia per fly with around 20 flies per experiment for each treatment. The experiment was repeated on three independent occasions. Raw data from individual ommatidia were analyzed non-parametrically using a Mann-Whitney U test to determine significance levels, with the STATVIEW software, version 4.53 (Abacus Concepts). A paired Student's t-test was used to compare the three experiment-average scores for each treatment.

### Quantification and Statistical Analysis of Aggregate Formation and Cell Death

Approximately 200 EGFP-positive cells per sample were counted for the proportion of cells with green fluorescent EGFP-HDQ74 aggregates, as described previously (2-4). If an EGFP-positive cell has one or many aggregates, the aggregate score is ‘one’. If an EGFP-positive cell does not have any aggregate, the aggregate score is ‘zero’. For example, the statement ‘calpastatin significantly reduced EGFP-HDQ74 aggregates’ means that calpastatin significantly reduced the proportion of EGFP-positive cells with EGFP-HDQ74 aggregates. Only EGFP-positive cells were counted so that we count only the transfected cells. Nuclei were stained with DAPI and those showing apoptotic morphology (fragmentation or pyknosis) were considered abnormal. These criteria are specific for cell death, which highly correlate with propidium iodide staining in live cells (16). Analysis was performed using Nikon Eclipse E600 fluorescence microscope (plan-apo 60×/1.4 oil immersion lens at room temperature) with observer blinded to identity of slides. Slides were coded and the code was broken after completion of experiment. All experiments were done in triplicate at least twice.

### Assessment of Autophagy by LC3

When autophagy is induced, the microtubule-associated protein 1 light chain 3 (LC3) is processed post-translationally into LC3-I, and then to LC3-II, which associates with autophagosome membranes (5). Quantification of the number of cells with LC3-positive vesicles or LC3-II levels (versus actin) allows for a specific and sensitive assessment of autophagosome number in large numbers of cells (17).

Furthermore, as EGFP-LC3 overexpression does not affect autophagic activity, the numbers of EGFP-LC3 vesicles have frequently been used to assess autophagosome number (18). For analysing the cells with EGFP-LC3 vesicles, we considered an EGFP-positive cell as having a score of ‘zero’ if there were 5 or fewer vesicles (as cells have basal levels of autophagy) and cells scored ‘one’ if they had >5 LC3-positive vesicles (4). Analysis was performed using Nikon Eclipse E600 fluorescence microscope (plan-apo 60×/1.4 oil immersion lens at room temperature) with observer blinded to identity of slides. Slides were coded and the code was broken after completion of experiment. All experiments were done in triplicate at least twice.

### Immunocytochemistry

COS-7 cells were fixed with 4% paraformaldehyde. Primary antibodies included anti-LC3 (kind gift from T. Yoshimori) and anti-Phospho-4E-BP1 (Thr37/46). Standard fluorescence methods were used for detection and secondary antibodies used were goat anti-rabbit Alexa 488 Green and Alexa 594 Red (Cambridge Biosciences). Images were acquired on a Zeiss LSM510 META confocal microscope (63×1.4 NA plan-apochromat oil immersion) at room temperature using Zeiss LSM510 v3.2 software (Carl Zeiss, Inc.), and Adobe Photoshop 6.0 (Adobe Systems, Inc.) was used for subsequent image processing.

### Western Blot Analysis

Cell pellets were lysed on ice in Laemmli buffer (62.5 mM Tris-HCl pH 6.8, 5% β-mercaptoethanol, 10% glycerol, and 0.01% bromophenol blue) for 30 min in the presence of protease inhibitors (Roche Diagnostics). Samples were subjected to SDS-polyacrylamide gel electrophoresis and proteins were transferred to nitrocellulose membranes (Amersham Pharmacia Biotech). Primary antibodies used include anti-EGFP (8362-1, Clontech), anti-HA (12CA15, Covance), anti-mTOR (2972), anti-Phospho-mTOR (Ser2448) (2971), anti-p70 S6 Kinase (9202), anti Phospho-p70 S6 Kinase (Thr389) (9206), anti-4E-BP1 (9452), anti-Phospho-4E-BP1 (Thr37/46) (9459), all from Cell Signaling Technology, anti-calpain small subunit (μ- or m-calpain) (MAB3083, Chemicon), anti-calpain 1 (MAB3104, Chemicon), anti-calpain 2 domain III (208755, Calbiochem), anti-Gsα (06-237, Upstate), anti-LC3 (NB100-2220, Novus), anti-LC3, anti-actin (A2066, Sigma) and anti-tubulin (Clone DM 1A, Sigma). Blots were probed with anti-mouse or anti-rabbit IgG-HRP (Amersham) and visualised using ECL or ECL Plus detection kit (Amersham).

### Measurement of Stored Intracellular Ca²⁺

Following 24 h treatment with 2.5 μM thapsigargin alone or in combination with 10 μM calpastatin or 50 μM ALLM (as previously described), COS-7 cells were pre-incubated with 1 μM Fura2-AM and an equal volume of Pluronic F127 (both Molecular Probes) for 45 min at room temperature in HEPES-buffered saline (HBS) containing 2 mM CaCl₂ and stored on ice. Aliquots of cells (5×10⁵/run) were resuspended in nominally Ca²⁺-free HBS. Stored intracellular Ca²⁺ was released into cytoplasm by adding 10 μM ionomycin. Experiments on PC12 cells were carried out following a similar loading regime for cells (but using 4 μM Fura-2-AM) and were performed using HBS containing 2 mM Ca²⁺ as extracellular solution. All experiments were performed at 37° C. and quantification of intracellular Ca²⁺ was achieved by monitoring Fura-2 fluorescence using a Cairn Spectrophotometer as described previously (19).

### RNA Interference

SMARTpool siRNA (pool of four different siRNA duplexes) against calpain 1 and calpain 2 and against GNAS (Dharmacon) were used for knockdown of the respective calpains and Gsα. Control siRNA (Ambion) or siGLO RISC-free siRNA (Dharmacon), which is fluorescent and does not target any human or mouse genes, was used as a control. HeLa cells were transfected with siRNAs (200 nM per well) for 96 h using Oligofectamine (Invitrogen) according to manufacturer's protocol. For experiment with EGFP-LC3, HeLa cells were transfected with siGLO alone or in combination with calpain 1 siRNA or calpain 2 siRNA (total 320 nM per well in 1:3 ratio) for 96 h followed by transfection with EGFP-LC3 (1 μg per well) using LipofectAMINE PLUS (Invitrogen) for 4 h. Cells were fixed after 2 h and analysed by fluorescence microscopy. For experiment with EGFP-HDQ74, HeLa cells were transfected with EGFP-HDQ74 (1.5 μg per well) along with siGLO or control siRNA in presence or absence of calpain 1 siRNA, calpain 2 or GNAS siRNA (1:3 ratio as above) for 96 h using LipofectAMINE 2000 (Invitrogen) according to manufacturer's protocol. Cells were fixed after transfection and analysed by fluorescence microscopy. The total amount of siRNA in the above experiments is identical in control and calpain/Gsα knockdown cells.

### Statistical Analysis by Odds Ratio

Pooled estimates for the changes in aggregate formation, cell death or EGFP-LC3 vesicles, resulting from perturbations assessed in multiple experiments, were calculated as odds ratios with 95% confidence intervals [Odds ratio of aggregation=(percentage of cells expressing construct with aggregates in perturbation conditions/percentage of cells expressing construct without aggregates in perturbation conditions)/(percentage of cells expressing construct with aggregates in control conditions/percentage of cells expressing construct without aggregates in control conditions)]. Odds ratios were considered to be the most appropriate summary statistic for reporting multiple independent replicate experiments of this type, because the percentage of cells with aggregates under specified conditions can vary between experiments on different days, whereas the relative change in the proportion of cells with aggregates induced by an experimental perturbation is expected to be more consistent. We have used this method frequently in the past to allow analysis of data from multiple independent experiments (2, 4, 11, 16, 20). Odds ratios and p values were determined by unconditional logistical regression analysis, using the general log-linear analysis option of SPSS 9 software (SPSS, Chicago). ***, p<0.001; **, p<0.01; *, p<0.05; NS, Non-significant.

### Results

Screen for Drugs that Enhance the Clearance of Aggregate-Prone Proteins

We screened for autophagy enhancers using a library of 250 compounds that had previously been used in man without major toxic side effects. We first assayed clearance of A53T α-synuclein, a known autophagy substrate, in stable PC12 cells (4, 15). A53T α-synuclein clearance was enhanced by known autophagy inducers like rapamycin and valproate (4, 16), five drugs that antagonise L-type Ca²⁺ channel activity (verapamil, nimodepine, loperamide, nitrendipine and amiodarone) and the following hits: minoxidil (an ATP-sensitive K⁺ channel agonist) and clonidine (binds to α2-adrenergic and type I imidazoline receptors and activates Gᵢ-protein signalling pathways) (FIG. 1). (±)-Bay K8644 (an L-type Ca²⁺ channel agonist (17)) retarded A53T α-synuclein clearance (FIG. 1).

We prioritised our validation studies on minoxidil and clonidine. These compounds enhanced soluble mutant huntingtin exon 1 with 74Q (EGFP-HDQ74) clearance in stable PC12 cells (which show no toxicity at these time points) and reduced its aggregation and toxicity in SK-N-SH cells, whereas (±)-Bay K8644 had opposite effects (FIGS. 2 & 3).

### Clonidine, Minoxidil and Verapamil Increase Autophagy to Reduce Mutant Huntingtin Aggregates

We assessed autophagosome numbers using the microtubule-associated protein 1 light chain 3 (LC3) (22). LC3 is processed post-translationally into LC3-I, then converted to LC3-II, the only known protein that specifically associates with autophagosome membranes (23). LC3-positive vesicle numbers or LC3-II levels (versus actin) correlate with autophagosome numbers (22). LC3-II levels were increased by clonidine, minoxidil and a representative Ca²⁺ channel antagonist, verapamil, providing indication of enhanced autophagy (FIG. 4). We confirmed that these drugs (and Bay K8644) influenced huntingtin aggregation in an autophagy-dependent manner as they had no effects in autophagy-deficient mouse embryonic fibroblasts (MEFs) that have a knockout of the key autophagy gene atg5 (Atg5^{-/-}), while their effects in wild-type MEFs (Atg5^{+/+}) mirrored that in SK-N-SH cells (FIG. 5).

### Clonidine Signals Through the Imidazoline Type 1 Receptor

Clonidine binds both α2-adrenergic and imidazoline-1 receptors. When clonidine binds α2-adrenergic receptors, Gᵢ signalling pathways are activated that reduce cAMP levels by inhibiting adenylyl cyclase (24, 25). Imidazoline-1 receptor binding also reduces cAMP levels, although whether this is mediated by G-proteins is disputed (26, 27). As clonidine had beneficial effects in both PC12 cells (which do not have α2-adrenergic receptors) and SK-N-SH cells (which have these receptors), we confirmed that rilmenidine, which binds imidazoline receptors at far higher affinities than the α2-adrenergic receptors, could also enhance A53T α-synuclein clearance and decrease EGFP-HDQ74 aggregation in these cells, respectively (FIGS. 6, 7 and 8).

Imidazoline-1 Receptor Agonists Signal to Phospholipase C Via the cAMP/EPAC/Rap2B Pathway

Clonidine and rilmenidine act by reducing cAMP levels, as the adenylyl cyclase inhibitor 2′5′-dideoxyadenosine (2′5′ddA) enhanced A53T α-synuclein clearance and decreased EGFP-HDQ74 aggregation, while the cAMP analogue (dibutyryl cAMP) and the adenylate cyclase activator (forskolin) had opposite effects. Furthermore, LC3-II levels were increased by rilmenidine and 2′5′DDA (FIGS. 6, 7 and 8).

cAMP can signal to at least three different pathways; protein kinase A (PKA), Epac (a guanine nucleotide exchange factor) and through cyclic nucleotide activated ion channels (28). The Epac-specific cAMP analogue, 8-CPT-2-Me-cAMP, slowed A53T α-synuclein clearance and enhanced mutant huntingtin aggregation, while the PKA-specific cAMP analogue, 6-Bnz-cAMP, had no effects (FIG. 9). Likewise, KT 5720, a PKA inhibitor, did not enhance A53T α-synuclein clearance (FIG. 10). Henceforth, we focused on the Epac pathway and tested its downstream components.

Epac activates the small GTPase Rap2B, which in turn activates the ubiquitously expressed phospholipase C (PLC)-ε isoform (29), which hydrolyses PIP₂ to form IP₃ and diacylglycerol. We have also confirmed Rap2B activation in PC12 cells treated with forskolin. This established pathway from Gᵢ-coupled receptors to PLC-ε (30, 31) was therefore a strong candidate autophagy regulator. Consistent with this hypothesis, dominant-negative Rap2B decreased EGFP-HDQ74 aggregation and increased LC3-II levels, while overexpression of PLC-ε enhanced EGFP-HDQ74 aggregation and decreased LC3-II levels (FIGS. 11 and 12). Consistent with a link between Epac and Rap2B, the deleterious effects of the Epac-specific cAMP analogue (8-CPT-2-Me-cAMP) on huntingtin aggregation, were abrogated by dominant-negative Rap2B (FIG. 13). Furthermore, overexpression of IP₃ kinase, which reduces IP₃ by conversion to IP₄, decreased EGFP-HDQ74 aggregation/toxicity (FIG. 14).

### Phospholipase C/IP₃, L-Type Ca²⁺ Channel Agonists/Antagonists and Minoxidil Regulate Calpain Activity

Minoxidil decreases whole-cell L-type Ca²⁺ channel currents in a dose-dependent manner (33) and was found to decrease thapsigargin-induced calpain activation. Minoxidil may therefore act on autophagy by inhibition of calpain via the same pathway as L-type Ca²⁺ channel blockers. In contrast to minoxidil, K⁺_{ATP} channel blockers, such as quinine sulphate (66) and tolazamide (67), slow clearance of autophagy substrates (FIG. 15).

### Gₛα is a Calpain Substrate Regulating Autophagy

The alpha subunit of heterotrimeric G-proteins (Gₛα) increases its activity after calpain cleavage (37) and enhances adenylyl cyclase activity. The hallmark 20 kDa Gₛα cleavage product was found to appear in our cells over-expressing active m-calpain. Activation of Gₛα with its natural ligand, pituitary adenylate cyclase-activating polypeptide (PACAP) retarded A53T α-synuclein clearance (FIG. 16). This effect was not blocked by calpastatin (as PACAP activation can occur without Gₛα cleavage) but was abolished by inhibition of its downstream target, adenyl cyclase (FIG. 17). PACAP also enhanced EGFP-HDQ74 aggregation (FIG. 18).

Conversely, siRNA knockodown of Gₛα or treatment with its chemical inhibitor NF449 decreased EGFP-HDQ74 aggregation (FIG. 19) and increased LC3-II levels. The increase in LC3-II levels with G_{sα} knockdown by siRNA was also seen in cells treated with bafilomycin A1, providing indication that knockdown of G_{sα} increases autophagosome synthesis. G_{sα} mediates many of the autophagy-related effects of calpains on EGFP-HDQ74 aggregation, as the increased aggregation mediated by overexpression of constitutively active m-calpain was abrogated by G_{sα} siRNA, and the beneficial effects of calpain inhibition are not further enhanced in calpastatin-treated cells with G_{sα} knockdown. Adenylyl cyclase inhibition by 2′5′ ddA decreased the enhanced EGFP-HDQ74 aggregation mediated by overexpression of wild-type or constitutive active m-calpain (FIG. 20), in a manner similar to that observed with PACAP.

Atg5 may be cleaved by calpains in certain cell death contexts. However, we observed no reduction in full-length Atg5 and no obvious cleavage products in cells treated with thapsigargin and no increase in Atg5 levels with calpastatin.

Therapeutic Potential of mTOR-Independent Autophagy Pathway in HD Models

In order to test whether compounds acting via the pathway identified in this screen were potentially relevant to therapy, we first tested them in a fly HD model. Flies expressing a mutant huntingtin fragment with 120Q in the photoreceptors exhibit photoreceptor degeneration that is not observed in flies expressing the wild-type protein with 23Q (40). The compound eye of Drosophila consists of many ommatidia, each composed of eight photoreceptor neurons with light-gathering parts called rhabdomeres, seven of which can be visualised under a light microscope using the pseudopupil technique (41). Neurodegeneration in the HD flies is progressive and is associated with a decrease in the number of visible rhabdomeres in each ommatidium with time (40). This process is attenuated with drugs that inhibit L-type Ca²⁺ channels (verapamil, nimodepine, loperamide) and drugs that act on the cAMP arm of the pathway (clonidine, and tyramine, that binds to tyramine receptors that activate Gᵢ) (FIG. 21A-21E). Valproate [that acts to induce autophagy by reducing IP₃ levels (16), among other activities that may be relevant to HD], also had protective effects.

Therapeutic Potential of mTOR-Independent Autophagy Pathway for Tuberculosis

As macrophages can clear mycobacteria via autophagy, we confirmed the potential utility of the pathway for this condition. We have described by showing that calpastatin and 2′5′ ddA reduced the numbers of mycobacteria in primary macrophages (FIG. 22), with an established assay (12). Similar effects are seen with rapamycin, which are blocked by 3-MA (FIG. 22). We have further shown the clinical potential of this pathway by using carbemazepine [a commonly used drug that lowers IP3 and induces autophagy (8)] (FIG. 22).

Our compound screen has identified a pathway regulating autophagy that provides many possibilities for therapeutic intervention (FIG. 23). In mammalian cells, autophagy is inhibited by L-type Ca²⁺ channel agonists, such as (±)-Bay K8644, which increases intracellular Ca²⁺ and activates calpain. Activation of calpain inhibits autophagy, L-type Ca²⁺ channel antagonists (Verapamil, Loperamide, Nimodipine, Nitrendipine, Amiodarone) or ATP-sensitive K⁺ channel agonists (Minoxidil) which decrease intracellular Ca²⁺ and prevent calpain activation, or calpain inhibitors (Calpastatin, ALLM, Calpeptin) induce autophagy, thereby enhancing the clearance of aggregate-prone mutant proteins and reducing their toxicity. Similarly, autophagy can be induced by clonidine and rilmenidine, which decrease the levels of cAMP. One target of cAMP is Epac which subsequently activates Rap2B, a small G-protein that can activate PLC-ε resulting in the production of IP₃ and consequent Ca²⁺ release from the ER. This Ca²⁺ release can activate calpains which through cleavage of Gsα can activate adenylate cyclase to increase cAMP levels thereby produce a self-propagating loop. In this model, compounds that inhibit either L-type Ca²⁺ channels, calpain or cAMP-coupled receptors or activate ATP-sensitive K⁺ channels, interfere with this loop to up regulate autophagy. Such compounds have therefore, the potential to be possible therapeutic targets for treatment of neurodegenerative disorders such as HD, PD and AD, as well as pathogen infections such as tuberculosis and viral infections.

Thus, compounds which inhibit L-type Ca²⁺ channels or which activate ATP sensitive K⁺ channels enhance the clearance of mutant proteins by inducing autophagy through calpain inhibition. Gsα provides a link between these two pathways as it is activated following calpain cleavage leading to increased cAMP levels. Interestingly, mutant huntingtin aggregation can be significantly inhibited by genetic knockdown of Gsα. This is the first demonstration of the importance of the Epac pathway in autophagy regulation.

Modulation of the Epac pathway may be valuable as an autophagy-inducing treatment for a range of diseases. Here we have provided proof-of-principle for drugs acting either via the cAMP/EPAC/PLC or ATP sensitive K⁺ channel routes having beneficial effects in cell-based, and fly models of HD. Furthermore, our studies provide indication that rapamycin/mTOR-independent, non-immunosuppressive drugs represent a new way of treating tuberculosis. Our data also provide indication that such drugs may have added efficacy for neurodegenerative diseases in combination with rapamycin, providing a new direction for combinatorial treatment of disorders like HD by enhancing autophagy through two different routes. Combination therapy with more moderate cAMP/EPAC/PLC and mTOR inhibition may be safer for long-term treatment compared to using higher doses of either compound that result in more severe inhibition of a single pathway. This strategy may allow a larger safety window before toxicity resulting from non-autophagy-related effects of each drug is observed.

### REFERENCES

1. D. C. Rubinsztein, Trends Genet 18, 202 (April, 2002).
2. J. Gafni, L. M. Ellerby, J Neurosci 22, 4842 (Jun. 15, 2002).
3. B. Ravikumar et al Hum Mol Genet 11, 1107 (May 1, 2002).
4. J. L. Webb, B. Ravikumar, J. Atkins, J. N. Skepper, D. C. Rubinsztein, J Biol Chem 278, 25009 (Jul. 4, 2003).
5. Z. H. Qin et al., Hum Mol Genet 12, 3231 (Dec. 15, 2003).
6. A. Iwata et al., PNAS USA 102, 13135 (Sep. 13, 2005).
7. Z. Berger et al., Hum Mol Genet 15, 433 (Feb. 1, 2006).
8. M. Shibata et al., J Biol Chem (March 6, In press).
9. B. Ravikumar et al., Nat Genet 36, 585 (June, 2004).
10. I. Nakagawa et al., Science 306, 1037 (Nov. 5, 2004).
11. M. Ogawa et al., Science 307, 727 (Feb. 4, 2005).
12. M. G. Gutierrez et al., Cell 119, 753 (Dec. 17, 2004).
13. B. Levine, J. Yuan, J Clin Invest 115, 2679 (October, 2005).
14. D. D. Sarbassov et al Curr. Opin. Cell Biol. 17, 596 (December, 2005).
15. A. M. Cuervo, et al Science 305, 1292 (Aug. 27, 2004).
16. S. Sarkar et al., J Cell Biol 170, 1101 (Sep. 26, 2005).
17. D. A. Greenberg et al Brain Res. 305, 365 (1984).
18. J. Church et al Mol. Pharmacol. 45, 747 (1994).
19. G. H. Hockerman et al Ann. Rev. Pharmacol. Toxicol. 37, 361 1997.
20. A. H. Sharp, et al J. Biol. Chem. 262, 12309 (1987).
21. J. Striessnig et al., FEBS Lett. 212, 247 (1987).
22. N. Mizushima, Int J Biochem Cell Biol 36, 2491 (December, 2004).
23. Y. Kabeya et al., Embo J 19, 5720 (Nov. 1, 2000).
24. N. N. Osborne, Brain Res 553, 84 (Jul. 5, 1991).
25. B. Bouscarel, et al Eur J Pharmacol 107, 223 (Jan. 2, 1985).
26. D. Felsen et al., J Pharmacol Exp Ther 268, 1063 (February, 1994).
27. H. Greney et al., Mol Pharmacol 57, 1142 (June, 2000).
28. R. Kopperud, et al FEES Lett 546, 121 (Jul. 3, 2003).
29. G. G. Kelley, et al, Embo J 20, 743 (Feb. 15, 2001).
30. M. Schmidt et al., Nat Cell Biol 3, 1020 (November, 2001).
31. F. vom Dorp et al., Cell Signal 16, 921 (August, 2004).
32. D. E. Goll et al Physiol Rev 83, 731 (July, 2003).
33. S. Hayashi et al J Pharmacol Exp Ther 265, 1527 (June, 1993).
34. D. J. Klionsky, S. D. Emr, Science 290, 1717 (Dec. 1, 2000).
35. S. Sarkar et al, J Biol Chem (Dec. 20, 2006).
36. B. Ravikumar et al., Nat Genet 37, 771 (July, 2005).
37. K. Sato-Kusubata et al Biochem J 347 Pt 3, 733 (May 1, 2000).
38. B. D. Manning et al Trends Biochem Sci 28, 573 (November, 2003).
39. J. Gafni et al., J Biol Chem 279, 20211 (May 7, 2004).
40. G. R. Jackson et al., Neuron 21, 633 (September, 1998).
41. N. Franceschini, in Information processing in the visual system of Drosophila R. Wehner, Ed. (Berlin: Springer, 1972) pp. 75-82.
42. Y. V. Budovskaya et al J Biol Chem 279, 20663 (May 14, 2004).
43. I. Holen et al Eur J Biochem 236, 163 (Feb. 15, 1996).
44. P. B. Gordon et al Biol Chem 268, 26107 (Dec. 15, 1993).
45. F. Demarchi et al., J Cell Biol 175, 595 (Nov. 20, 2006).
46. D. J. Klionsky, Y. Ohsumi, Annu Rev Cell Dev Biol 15, 1 (1999).
47. S. L. Chan et al Neurobiol Dis 11, 2 (October, 2002).
48. M. M. Zeron et al., Neuron 33, 849 (Mar. 14, 2002).
49. T. S. Tang et al., Neuron 39, 227 (Jul. 17, 2003).
50. Y. Sun et al J Biol Chem 276, 24713 (Jul. 6, 2001).
51. S. Orrenius et al Nat Rev Mol Cell Biol 4, 552 (July, 2003).
52. Y. J. Kim et al., PNAS USA 98, 12784 (Oct. 23, 2001).
53. Y. Narain, A. et al J Med Genet 36, 739 (October, 1999).
54. A. Glading et al., Mol Cell Biol 24, 2499 (March, 2004).
55. E. T. W. Bampton et al Autophagy 1, 23 (2005).
56. N. P. Dantuma et al Nat Biotechnol 18, 538 (May, 2000).
57. A. Wyttenbach et al., Hum Mol Genet 10, 1829 (Aug. 15, 2001).
58. A. Wyttenbach et al., Hum Mol Genet 11, 1137 (May 1, 2002).
59. E. Ryder et al., Genetics 167, 797 (June, 2004).
60. N. Mizushima et al Mol Biol Cell 15, 1101 (March, 2004)
61. R. A. Floto et al., Nat Med 11, 1056 (October, 2005).
62. J. Carmichael et al J Biol Chem 277, 33791 (Sep. 13, 2002).
63. J. Gafni, L. M. Ellerby, J Neurosci 22, 4842 (Jun. 15, 2002).
64. O. Thastrup et al. Natl. Acad. Sci. U.S.A. 87, 2466 (1990).
65. S. Pattingre et al., Cell 122, 927 (Sep. 23, 2005).
66. Y. Kabeya et al EMBO J. 19, 5720-5728 (2000).
67. N. Osborne Brain Res. 553, 84-88 (1991).

1. A method of inducing or promoting autophagy in a cell comprising:
inhibiting or reducing the activity of the cAMP/EPAC/PLC pathway in said cell.

2. A method according to claim 1 wherein the activity of said pathway is reduced or inhibited by reducing the amount of cytosolic 3′, 5′ cyclic adenosine monophosphate (cAMP) in said cell.

3. A method according to claim 2 wherein the cell is contacted with a cAMP antagonist.

4. A method according to claim 3 wherein the cAMP antagonist is selected from the group consisting of clonidine, rilmenidine, tyramine, morphine, baclofen, mastoparan, propranolol, bupivacain, N-dodecyl lysinamide, a compound shown in FIG. 24 or FIG. 25 or an analogue or derivative thereof.

5. A method according to claim 3 wherein the cAMP antagonist is an antagonist of Gₛα and/or PACAP.

6. A method according to claim 5 wherein the antagonist of Gₛα and/or PACAP is suramin, NF449 or NF503.

7. A method according to claim 3 wherein the cAMP antagonist is an antagonist of adenylate cyclase.

8. A method according to claim 7 wherein the adenylate cyclase antagonist is 2′5′ dideoxyadenosine.

9. A method according to claim 1 wherein the cell is contacted with an antagonist of one or more components of the cAMP/EPAC/PLC pathway.

10. A method according to claim 9 wherein the cell is contacted with an antagonist of Epac1, Rap2B and PLC-ε.

11. A method according to claim 2 wherein the cell is contacted with an ATP-sensitive K⁺ channel agonist.

12. A method according to claim 11 wherein the ATP-sensitive K⁺ channel agonist is minoxidil, pinacidil, cromakalim or an analog or derivative thereof.

13. A method according to any one of claims 1 to 12 wherein the cell is further contacted with an mTOR inhibitor.

14. A method according to claim 13 wherein the mTOR inhibitor is a rapamycin macrolide.

15. A method according to any one of the preceding claims comprising determining the level of autophagy in the cell.

16. A method according to any one of the preceding claims wherein the cell is comprised in an individual having a neurodegenerative disorder or pathogen infection.

17. Use of an agent which inhibits or reduces the activity of the cAMP/EPAC/PLC pathway in a cell in the manufacture of a medicament for use in the induction of autophagy in a cell.

18. Use according to claim 17 wherein the agent reduces the amount of cytosolic 3′, 5′ cyclic adenosine monophosphate (cAMP) in said cell.

19. Use according to claim 18 wherein the agent is a cAMP antagonist.

20. Use according to claim 19 wherein the cAMP antagonist is selected from the group consisting of clonidine, rilmenidine, tyramine, morphine, baclofen, mastoparan, propranolol, bupivacain, N-dodecyl lysinamide, a compound shown in FIG. 19 or FIG. 20 or an analogue or derivative thereof.

21. Use according to claim 19 wherein the cAMP antagonist is an antagonist of Gₛα and/or PACAP.

22. Use according to claim 21 wherein the antagonist of Gₛα, and/or PACAP is NF449.

23. Use according to claim 19 wherein the cAMP antagonist is an antagonist of adenylate cyclase.

24. Use according to claim 23 wherein the adenylate cyclase antagonist is 2′5′ dideoxyadenosine.

25. Use according to claim 18 wherein the agent is an antagonist of one or more components of the cAMP/EPAC/PLC pathway.

26. Use according to claim 25 wherein the cell is contacted with an antagonist of Epac1, Rap2B and PLC-ε.

27. Use according to claim 18 wherein the agent is an ATP-sensitive K⁺ channel agonist.

28. Use according to claim 27 wherein the ATP-sensitive K⁺ channel agonist is minoxidil, pinacidil, cromakalim or an analog or derivative thereof.

29. Use according to any one of claims 17 to 28 wherein the cell is further contacted with an mTOR inhibitor.

30. Use according to claim 29 wherein the mTOR inhibitor is a rapamycin macrolide.

31. Use according to any one of claims 17 to 30 wherein the cell is comprised in an individual having a neurodegenerative disorder or pathogen infection.

32. A method or use according to claim 16 or claim 31 wherein the neurodegenerative disorder is a protein aggregation disorder.

33. A method or use according to claim 32 wherein the disease is a tauopathy.

34. A method or use according to claim 33 wherein the disorder is Alzheimer's disease.

35. A method or use according to claim 32 wherein the disorder is a codon reiteration mutation disorder.

36. A method or use according to claim 35 wherein the disorder is a polyA expansion disorder

37. A method or use according to claim 35 wherein the disorder is a polyQ expansion disorder.

38. A method or use according to claim 37 wherein the polyQ expansion disorder is selected from the group of Huntington's disease, spinocerebellar ataxias types 1, 2, 3, 6, 7 and 17, spinobulbar muscular dystrophy and dentatorubral pallidoluysian atrophy.

39. A method or use according to claim 32 wherein the disease is a α-synucleinopathy.

40. A method or use according to claim 39 wherein the α-synucleinopathy is selected from the group of Parkinson's Disease, LB variant Alzheimer's disease and LB dementia.

41. A method or use according to claim 16 or claim 31 wherein the pathogen infection is a bacterial infection.

42. A method or use according to claim 41 wherein the bacterial infection is a mycobacterial infection.

43. A method or use according to claim 42 wherein the mycobacterial infection is tuberculosis.

44. A method or use according to claim 41 wherein the bacterial infection is a streptococcal infection.

45. A method or use according to claim 16 or claim 31 wherein the pathogen infection is a viral infection.

46. A method or use according to claim 45 wherein the viral infection is a herpes simplex virus or Sindbis virus infection.

47. A pharmaceutical composition comprising an agent which inhibits or reduces the activity of the cAMP/EPAC/PLC pathway in a cell, an mTOR inhibitor and a pharmaceutically acceptable excipient.

48. An agent which inhibits or reduces the activity of the cAMP/EPAC/PLC pathway in a cell for use in the treatment of a neurodegenerative disorder or pathogen infection.

49. A method of identifying and/or obtaining a compound which induces autophagy in a cell comprising,
determining the ability of a test compound to reduce the activity of the cAMP/EPAC/PLC pathway in a cell.

50. A method according to claim 49 wherein said ability is determined by determining the ability of a test compound to reduce the level or amount of cAMP in the cell.

51. A method according to claim 50 wherein said ability is determined by determining the ability of a test compound to reduce the activity of adenylate cyclase in the cell.

52. A method according to claim 50 wherein said ability is determined by determining the ability of a test compound to reduce the activity of Gₛα and/or PACAP in the cell.

53. A method according to claim 49 wherein said ability is determined by determining the ability of a test compound to reduce the activity of one or more components of the cAMP/ERAC/PLC pathway in the cell.

54. A method according to claim 53 wherein the one or more components are selected from the group consisting of Epac1, Rap2B and PLC-ε.

55. A method according to claim 49 wherein said ability is determined by determining the ability of a test compound to reduce the activity of an ATP-sensitive K⁺ channel in the cell.

56. A method according to any one of claims 49 to 55 wherein a reduction in the activity of the cAMP/EPAC/PLC pathway in the cell in the presence relative to the absence of test compound is indicative that the compound is useful in inducing autophagy.

57. A method according to any one of claims 49 to 56 further comprising contacting said test compound with a cell and determining the level or amount of autophagy in said cell.

58. A method according to any one of claims 49 to 56 comprising identifying the test compound as compound which induces autophagy in a cell, said compound being useful in the treatment of a neurodegenerative disorder or a pathogenic infection.

59. A method according to according to claim 58 comprising isolating and/or purifying the test compound.

60. A method of decreasing autophagy in a cell comprising;
activating or increasing the activity of the cAMP/EPAC/PLC pathway in said cell.
